(19) United States
(12) Patent Application Publication
(10) Pub. No.: US 2024/0368569 A1
(43) Pub. Date: Nov. 7, 2024

(54) ENGINEERED HUMAN EXTRACELLULAR DNASE ENZYMES FOR DRUG CANDIDATE SELECTION

(71) Applicant: Neutrolis, Inc., Cambridge, MA (US)

(72) Inventor: Tobias A. FUCHS, Wellesley, MA (US)

(21) Appl. No.: 18/655,606

(22) Filed: May 6, 2024

Related U.S. Application Data
(63) Continuation of application No. 17/427,974, filed on Aug. 3, 2021.
(60) Provisional application No. 62/800,790, filed on Feb. 4, 2019.

(51) Int. Cl. C12N 9/16; A61K 38/46; A61K 47/60; C07K 14/765

(52) U.S. Cl. CPC C12N 9/16; A61K 38/465; A61K 47/60; C07K 14/765; C12Y 301/21001; C07K 2319/31

(57) ABSTRACT

The present disclosure provides a library of engineered DNASE proteins (including DNASE1, DNASE1-LIKE 1, DNASE1-LIKE 2, DNASE1-LIKE 3, DNASE2A, DNASE2B) that allows to select drug candidates for developing therapeutics for treating conditions characterized by neutrophil extracellular trap (NET) accumulation and/or release. In accordance with the invention, the selected DNase variant has improved properties, including properties amenable to clinical development, including manufacturing, toxicology, pharmacokinetic, and/or use in therapy.

### PRIORITY

This application is a continuation of U.S. Non-Provisional application Ser. No. 17/427,974, filed Aug. 3, 2021, which is a 371 national phase application of PCT/US2020/016490, filed Feb. 4, 2020, which claims the benefit of, and priority to, U.S. Provisional Application No. 62/800,790, filed Feb. 4, 2019, which is hereby incorporated by reference in its entirety.

### SEQUENCE LISTING

The instant application contains a Sequence Listing which has been submitted in XML format via PatentCenter and is hereby incorporated by reference in its entirety. Said XML copy, created on Jul. 18, 2024, is named “NTR-006C1_Sequence_Listing.xml,” and is approximately 57,344 bytes in size.

### BACKGROUND

Inflammation is an essential host response to control invading microbes and heal damaged tissues. Uncontrolled and persistent inflammation causes tissue injury in a plethora of inflammatory disorders. Neutrophils are the predominant leukocytes in acute inflammation. During infections neutrophils generate neutrophil extracellular traps (NETs), lattices of DNA-filaments decorated with toxic histones and enzymes that immobilize and neutralize bacteria. However, excessive NET formation may harm host cells due to their cytotoxic, proinflammatory, and prothrombotic activity.

DNASE1 (D1) forms along with DNASE1-LIKE 1 (D1L1), DNASE1-LIKE 2 (D1L2) and DNASE1-LIKE 3 (D1L3), the DNASE1-protein family, a group of homologous secreted DNase enzymes. DNASE2A and DNASE2B form an additional group of homologous extracellular DNase enzymes. DNASE1- and DNASE2-protein family members are evolutionary conserved and expressed in various species, including humans. Recombinant human DNASE1- and DNASE2-protein family members provide drug candidates for NET-associated diseases, but the physical, enzymatic, toxicological, and pharmacokinetic properties of these enzymes are not ideal for clinical applications. Thus, there is a need for engineered DNASE enzymes for use in therapy that have improved properties, including properties amenable to clinical development, including manufacturing, toxicology, pharmacokinetic, and/or use in therapy.

### SUMMARY

The present invention provides candidates of engineered human extracellular DNASE proteins (e.g., variants of DNASE1 (D1), DNASE1-LIKE 1 (D1L1), DNASE1-LIKE 2 (D1L2), DNASE1-LIKE 3 Isoform 1 (D1L3), DNASE1-LIKE 3 Isoform 2 (D1L3-2), DNASE2A (D2A), and DNASE2B (D2B)) that are useful for treating conditions characterized by extracellular DNA, extracellular chromatin, and/or neutrophil extracellular trap (NET) accumulation and/or release. In accordance with aspects of the invention, DNASE variants described herein are more amenable to clinical development, including manufacturing, toxicology, pharmacokinetic, and/or use in therapy.

In some aspects, the invention provides a method for making a DNASE therapeutic composition for treating an extracellular chromatin or NET-associated disorder. The method comprises evaluating a plurality of extracellular DNASE variants for one or more characteristics, including enzymatic activity, nucleic acid substrate preference, potential for recombinant expression in prokaryotic or eukaryotic host cells, immunogenic potential in humans and animals, and pharmacodynamics in animal models. An extracellular DNASE variant is selected with the desired enzymatic, physical, and pharmacodynamics profile, and is formulated for administration to a patient, e.g., for either systemic or local administration.

In various embodiments, the DNASE variant evaluated and selected for use in therapy in accordance with embodiments of the invention comprise an amino acid sequence that is at least 80% identical to the enzyme defined by any one of SEQ ID NOS: 1 to 7, with one or more building block substitutions or C-terminal modifications as described herein. In some embodiments, the DNASE variant comprises an N-terminal or C-terminal fusion to a half-life extending moiety, such as albumin, transferrin, an Fc, or elastin-like protein.

In various embodiments, a selected DNASE variant is formulated with a pharmaceutically acceptable carrier for systemic, local, or topical administration.

In other aspects, the invention provides a method for treating a subject in need of extracellular DNA degradation, extracellular chromatin degradation, extracellular trap (ET) degradation and/or neutrophil extracellular trap (NET) degradation. The method comprises administering a therapeutically effective amount of the extracellular DNASE variant in accordance with the disclosure.

Other aspects and embodiments of the disclosure will be apparent from the following detailed description.

### BRIEF DESCRIPTION OF THE DRAWINGS

FIG. 1 illustrates the approach for engineering DNASE variants for therapeutic applications using Building Block Protein Engineering.

FIG. 2 shows an alignment of DNASE1-LIKE 3 Isoform 1 proteins from different species. Amino acids that are non-conserved in human DNASE1 are highlighted. Such non-conserved amino acids can be transferred to human DNASE1-LIKE 3 Isoform 1 for developing a variant for therapy. The DNASE1-LIKE 3 Isoform 1 proteins used for this analysis were Human DNASE1-LIKE 3 Isoform 1, UniProtKB: Q13609; NCBI Reference Sequence: NP_004935.1 (SEQ ID NO: 4); Pan troglodytes (Chimpanzee) DNase1L3 UniProtKB: A0A2I3RHL6 (and H2QMU7)(SEQ ID NO: 33); Papio anubis (Olive baboon) DNASE1L3, UniProtKB: A0A2I3NFJ3 (SEQ ID NO: 34); Mouse Dnase1l3, UniProtKB: 055070 (SEQ ID NO: 31); Rat DNase1L3, UniProtKB: 089107 (SEQ ID NO: 32); Oryctolagus cuniculus (Rabbit) DNase1L3, UniProtKB: GISE62 (SEQ ID NO: 35); Canis lupus familiaris (Dog) DNase1L3, UniProtKB: F1P9C1 (SEQ ID NO: 36); Sus scrofa (Pig) DNase1L3, UniProtKB: A0A287BI32 (SEQ ID NO: 37); Cavia porcellus (Guinea pig) DNase1L3, UniProtKB: A0A286XK50 (SEQ ID NO: 38); Bos taurus (Cow) DNase1L3, UniProtKB: F1MGQ1 (SEQ ID NO: 39); and Loxodonta africana (African elephant) DNase1L3, UniProtKB: G3SXX1 (SEQ ID NO: 40)

FIG. 3 shows an alignment of two members of the human DNASE1 proteins family, DNASE1-LIKE 1 and DNASE1-LIKE 3 Isoform 1. Amino acids that are conserved among human DNASE1-LIKE 1 (NCBI Reference Sequence: NP_006721.1; SEQ ID NO: 2) and DNASE1-LIKE 3 Isoform 1 (NCBI Reference Sequence: NP_004935.1; SEQ ID NO: 4) are highlighted. The non-conserved amino acids can be transferred from human DNASE1-LIKE 1 to DNASE1-LIKE 3 Isoform 1 or vice versa for developing variants for therapy, respectively.

FIG. 4 shows the concept of building block engineering of homologous proteins. The technology transfers single or multiple variable amino acids, which are flanked by conserved single or multiple variable amino acids, between a donor and recipient protein.

FIG. 5 shows an amino acid sequence alignment of DNase1 and DNase1L3 of mouse (SEQ ID NOs: 31 and 32), rat (SEQ ID NOs: 33 and 34), chimpanzee (SEQ ID NOs: 35 and 36), and human (SEQ ID NOs: 1 and 4). The N-terminal signal peptide, corresponding to N-terminal 22 amino acids of DNase1 is shown in light grey and conserved amino acids are highlighted in a darker shade of grey. Variable amino acids are not highlighted and serve as Building Blocks that can be transferred from DNase1 to DNase1L3 and vice versa. Abbreviations: AA, amino acid.

FIGS. 6A-FIG. 6B show lists of Building Blocks in human DNase1 (D1) and human DNase1L3 (D1L3). FIG. 6A shows amino acids that are conserved in D1 and D1L3, which serve as N- and C-anchors, respectively. Building blocks are variable amino acids in D1 and D1L3. Mutations that transfer Building Blocks from D1L3 to D1 are shown. FIG. 6B shows N- and C-anchors in D1L3. Mutations that transfer Building Blocks from D1 to D1L3 are listed. AA: amino acid.

FIG. 7 shows an application of the building block engineering of homologous proteins. The application uses as an initial screening step, the transfer of clusters of building blocks between a homologous donor and recipient protein. Additional optional steps are the transfer of individual building blocks, followed by the transfer of individual amino acids. In a final step (not shown), multiple amino acids, building blocks, and building block clusters may be combined to degenerate a chimeric enzyme.

FIG. 8 shows characterization of DNase1 variants (D1^{V}) featuring building blocks from DNase1L3 (D1L3). Zymography showed dsDNA degrading activity as dark circles. The dsDNA degrading activity correlates with the diameter. Samples without activity show the loading well as small black spot (e.g. Ctrl). Agarose gel electrophoresis (AGE) of DNA isolated from digested chromatin shows a shift from high-molecular weight DNA to lower or low-molecular weight DNA that correlates with chromatin degrading activity. Building block substitutions that cause an increase in chromatin degrading activity are highlighted in dark shade. Samples without such effect are shown in light shade. A DNase1 variant featuring the combination of building blocks 11, 12-14, 26, 41-48, and 49 shows similar chromatin degrading activity than wild-type DNase1L3.

FIG. 9 illustrates that the mutation Q282_S305delinsK in D1L3 Isoform 1 increases the activity to degrade high-molecular weight chromatin of DNASE1L3.

### DETAILED DESCRIPTION

As used herein, the term “neutrophil extracellular trap” or “NET” refers to any extracellular trap (“ET”) comprising extracellular DNA formed by cells such as, but not limited to, neutrophils, monocytes, macrophages, basophils, eosinophils, mast cells, cancer cells, injured cells (e.g., injured endothelial cells), and the like. Unless the context indicates otherwise, the terms NET and ET are used interchangeably herein.

The similarity of nucleotide and amino acid sequences, i.e. the percentage of sequence identity, can be determined via sequence alignments as known in the art. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). Exemplary algorithms are incorporated into the BLASTN and BLASTP programs of Altschul et al (1990) J. Mol. Biol. 215: 403-410. When utilizing BLAST programs, the default parameters of the respective programs are used.

In various aspects, the invention provides a protein engineering technology that is based on a transfer of a single amino acid or multiple-adjacent amino acids, termed “building block”, between two members of a protein family, such as DNase1 or DNase 2 protein family members to generate enzymatically active variants. A “building block” is defined by amino acids that are variable between two or more members of the DNase protein family. These variable amino acids are flanked by amino acids that are conserved between two or more members of the DNase-protein family (“anchors”). The variable single amino acid or multiple contiguous amino acids (“building blocks”) are exchanged between members of the DNase-protein family by implanting them between conserved single amino acid or multiple contiguous amino acids (“anchors”).

This approach is referred to herein as “building-block protein engineering.” Where three or more amino acids are transferred in a building block, up to ⅓ of the amino acids transferred may be further substituted. For example, where three to six amino acids are transferred as a building block, one or up to two resides may be further substituted. In some embodiments, four or more amino acids are transferred as a building block substitution, and up to 25% of the transferred amino acids are further substituted, e.g., with conservative or non-conservative amino acid modifications. For example, where four, eight, or twelve amino acids are transferred, one, two, or three amino acids (respectively) may be further substituted in the building block substitution.

The present invention provides candidates of engineered human extracellular DNASE proteins (e.g., variants of DNASE1 (D1), DNASE1-LIKE 1 (D1L1), DNASE1-LIKE 2 (D1L2), DNASE1-LIKE 3 Isoform 1 (D1L3), DNASE1-LIKE 3 Isoform 2 (D1L3-2), DNASE2A (D2A), and DNASE2B (D2B)) that are useful for treating conditions characterized by extracellular DNA, extracellular chromatin, and/or neutrophil extracellular trap (NET) accumulation and/or release. In accordance with aspects of the invention, DNASE variants described herein are more amenable to clinical development, including manufacturing, toxicology, pharmacokinetic, and/or use in therapy.

In some aspects, the invention provides a method for making a DNASE therapeutic composition for treating a NET-associated disorder or disorder characterized by pathological accumulation of extracellular chromatin. The method comprises evaluating a plurality of extracellular DNASE variants for one or more characteristics, including enzymatic activity, nucleic acid substrate preference, suitability for recombinant expression in prokaryotic or eukaryotic host cells, immunogenic potential in humans and animals, and pharmacodynamics in animal models. An extracellular DNASE variant is selected with the desired enzymatic, physical, and pharmacodynamics profile, and is formulated for administration to a patient, e.g., for either systemic or local administration.

In various embodiments, at least 5 or at least 10, or at least 20, or at least 50 extracellular DNASE variants are evaluated, with the variants selected from one or more of D1 variants, D1L1 variants, D1L2 variants, D1L3 isoform 1 variant, D1L3 isoform 2 variants, D2A variants, and D2B variants as described herein. As described herein, one or more (or all) variants may comprise at least one building block substitution, half-life extension moiety, and/or other mutation or variation described herein. In some embodiments, the method evaluates one or more D1L1 variants described herein. In some embodiments, the method evaluates one or more D1L2 variants described herein. In some embodiments, the method evaluates one or more D1L3 variants described herein. In some embodiments, the method evaluates one or more D1L3-2 variants described herein. In some embodiments, the method evaluates one or more D2A variants described herein. In some embodiments, the method evaluates one or more D2B variants described herein. In some embodiments, the method evaluates one or more D1 variants described herein.

In various embodiments, the invention provides a recombinant variant of human extracellular DNASE enzymes comprising one or more amino acid alterations resulting in an altered pH and temperature optimum, requirement for divalent cations for enzymatic activity, mechanisms of enzymatic inhibition (e.g. salt, divalent cations, actin, heparin, proteases), substrate affinity and specificity (e.g. single-stranded DNA, double-stranded DNA, chromatin, NETs, plasmid DNA, mitochondrial DNA), localization upon secretion (e.g. membrane-bound, extracellular matrix), localization signals (e.g. nuclear localization signal, membrane anchor), glycosylation sites, disulfide-bonds and unpaired cysteines, compatibility with GMP-compliant in vitro expression systems (e.g. bacteria. yeast, mammalian cells), compatibility with carriers (e.g. PEGylation, Fc fragment, albumin), compatibility with GMP-compliant purification methods (e.g. anion exchange resins, cation exchange resins), toxicological profile, tissue penetration, pharmacokinetics and pharmacodynamics. In accordance with this disclosure, candidate DNASE variants can be selected with desired properties for therapy.

In various embodiments, DNASE variants will comprise at least one building block substitution, using a Building Block Protein Engineering technology. The Building Block Engineering approach is described in PCT/US2018/047084, which is hereby incorporated by reference in its entirety. This approach involves providing a protein-protein alignment of donor and recipient DNASE enzymes, and identifying variable amino acid sequences for transfer (“building block”). The variable amino acid(s) are flanked by one or more conserved amino acids in the donor and recipient DNASE enzymes (upstream and downstream of the building block). These building blocks can be swapped between recipient and donor proteins, to produce a chimeric enzyme. The donor and recipient DNASE enzymes can be selected from members of the DNASE1- or DNASE2-protein family. Accordingly, for example, human DNASE1 and human DNASE1L1 can be selected as donor and recipient DNASE proteins, respectively. Alternatively, donor and recipient DNASE can be selected from a DNASE proteins that are expressed in different species. Accordingly, for example, bovine and human DNASE1 can be selected as donor and recipient DNASE proteins, respectively.

As used herein, when referring to sequence identity with wild-type extracellular DNASE enzymes, and unless stated otherwise, sequences refer to mature enzymes lacking the signal peptide. Further, unless stated otherwise, amino acid positions are numbered with respect to the full translated extracellular DNASE sequence, including signal peptide, for clarity. Accordingly, for example, reference to sequence identity to the enzyme of SEQ ID NO: 1 (human D1) refers to a percent identity with the mature enzyme having L23 at the N-terminus, reference to sequence identity to the enzyme of SEQ ID NO: 2 (human D1L1) refers to a percent identity with the mature enzyme having F19 at the N-terminus, reference to sequence identity to the enzyme of SEQ ID NO: 3 (human D1L2) refers to a percent identity with the mature enzyme having L22 at the N-terminus, reference to sequence identity to the enzyme of SEQ ID NO: 4 (human D1L3) refers to a percent identity with the mature enzyme having M21 at the N-terminus, reference to sequence identity to the enzyme of SEQ ID NO: 5 (human D1L3-2) refers to a percent identity with the mature enzyme having M21 at the N-terminus, reference to sequence identity to the enzyme of SEQ ID NO: 6 (human D2A) refers to a percent identity with the mature enzyme having C19 at the N-terminus, and reference to sequence identity to the enzyme of SEQ ID NO: 7 (human D2B) refers to a percent identity with the mature enzyme having A28 at the N-terminus.

The term “delins” refers to a deletion between two indicated amino acids, with an insertion of an amino acid or sequence of amino acids at the site of the deletion. For example, the notation E91_P92delinsSR means that the amino acids from E91 to P92 are deleted and the amino acids SR are inserted at the site of the deletion (e.g., the resulting amino acid sequence will have S91 and R92).

The term “ins” refers to an insertion of amino acids between two indicated amino acids. For example, the notation E91_P92insSR means that the amino acids SR are inserted between E91 and P92, resulting in the sequence E91, S92, R93, and P94.

The term “del” refers to a deletion of one amino acid or two and more amino acids between two indicated amino acids. For example, the notation E91del means that the amino acid E91 is deleted, whereas the notion E91_P93del means that the three amino acids from E91 and P93 are deleted.

The engineered variants of human extracellular DNASE enzymes may comprise one or more additional amino acid substitutions, additions (insertions), deletions, or truncations in the amino acid sequence of the human enzyme (SEQ ID NO: 1 to 7). Amino acid substitutions may include conservative and/or non-conservative substitutions. For example, “conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. “Conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In various embodiments, the DNASE1 (D1) variant evaluated and selected for use in therapy in accordance with embodiments of the invention comprises an amino acid sequence that is at least 80% identical to the enzyme defined by SEQ ID NO: 1, with one or more building block substitutions.

In some aspects, the building block substitutions are selected from non-human D1 proteins and result in variants of human D1, which feature one or more of the following mutations: K24R, I25M, Q31R, T32S, E35D, V44S, V44T, V44A, S45V, S45K, S45N, S45H, Q49K, Q49R, S52Q, S52R, R53L, I56V, A57V, L58V, V59I, S60T, T68V, D75N, N76E, N76K, N76T, N76E, N76Y, N76S, Q79R, Q79E, D80K, D80H, A81K, A81I, A81D, P82A, P82T, D83N, D83G, T84N, T84A, Y85F, H86R, Y87F, Y87H, V88I, V89I, V89A, N96K, N96R, N96S, S97T, R101Q, V105L, Y106F, D109S, Q110R, Q110K, A113V, A113I, S114L, S116T, Y108Q, Y108H, Y108L, P125S, N128T, T130S, N132S, N132A, A136S, I137V, R139K, F141S, F141H, S142C, R143P, R143H, F144Y, F144S, F144L, V147K, V147Q, R148Q, R148S, E149K, I152V, P154A, A157S, G160E, G160T, G160L, G160S, D161E, V163A, S164S, D167N, A168S, D175N, Q177W, Q177R, E178Q, E178K, E178H, G181D, G181H, E183Q, E183N, V185I, M186V, L187F, G194D, C195Y, R199T, R199A, R199S, P200S, P200A, P200T, P200L, Q202H, S204A, W209R, T210M, T210E, P212S, T213A, T213I, T213P, Q215K, Q215R, P219L, S221T, S221N, A226V, A226S, T227S, T227K, P228S, H230N, A232P, M241T, M241A, M241P, M241S, R244Q, G245D, G245A, G245H, G245R, G245S, D250N, D250S, D250E, D250G, L253V, L253A, L253M, N256D, A259V, A260E, Y261F, G262R, S264T, D265N, D265S, D265E, Q266E, L267M, L267T, Q269E, Q269L, M280T, M280A, K282R, K282A, K282T, K282insK, and K282insR.

In some embodiments, the building block substitutions to D1 are selected from human D1L1 and result in variants of human D1 which feature one or more of the following mutations: M1_G3del, K5_G8delinsHYPT, A12F, L14_Q15delinsAN, V21_K24delinsQAFR, A26C, I30A, T32_T36delinsRLTLA, M38_I47delinsVAREQVMDTL, Q49R, S52A, Y54C, A57_V59delinsMVL, R63V, H66_T68delinsSGS, V70_K72delinsIPL, D75_N76delinsRE, Q79delinsRF, A81_T84delinsGSGP, H86_V89delinsSTLS, E91_P92delinsPQ, N96_S97delinsST, K99M, R101T, L103_V105delinsVYF, P108_D115delinsSHKTQVLS, Y118V, D120N, G122_N128delinsED, T130V, A136_R139delinsFVAQ, F141_I152delinsSLPSNVLPSLVL, A157_A158delinsTT, G160_A164delinsKAVEK, I166_D167delinsLN, Y173F, D175E, G177_E179delinsQSK, M186L, G194D, S196_R207delinsASLTKKRLDKLE, W209R, S211E, T213G, Q215H, L217V, P219A, S221_A222delinsGE, A226_P228delinsVRAS, A232T, I236V, V238_A239delinsLH, M241_L243delinsERC, G245 S251delinsSLLHT, L253_P254delinsAA, N256D, Q259_G262delinsPTSQG, S264_L267delinsTEEE, Q269_A270delinsLN, M280E, and K282delinsKLSQAHSVQPLSLTVLLLLSLLSPQLCPAA.

In certain embodiments, the building block substitutions to D1 are selected from human D1L2 and result in variants of human D1 which feature one or more of the following mutations: R2G, M4_K5delinsPRA, G8A, L11W, A14E, L16_Q18delinsA, A20_S22delinsTAA, K24R, A26G, T32S, E35_T36delinsDS, M38V, N40_V44delinsDPACG, Y46I, V48_Q49delinsAK, S52_R53delinsAG, I56L, S65_H66delinsPD, T68S, S70_A71delinsGK, L74_L77delinsMEQI, Q79_T84delinsSVSEHE, H86_Y87delinsSF, V89S, E91Q, D95_Q96delinsNS, R101M, P108K, Q110A, A113V, S116T, Y118L, P119D, G122_N128delinsPE, T130V, N132S, A136_I137delinsFV, R139K, F141_F144delinsSAPG, E146_V153delinsGERAPPLPSRRALTPPLPAAAQNLVLI, G160_D161delinsHQ, Q177_E178delinsID, L182_E182delinsTD, V185_M188delinsMLFL, G194D, P200_Q202delinsAQD, S204_S205delinsAA, W209_T210delinsRS, P212_T213delinsEV, Q230K, A226_H230delinsVGNSD, V239_A240delinsAC, M241_L242delinsAR, G245_V248delinsRSLK, D250Q, L253_N256delinsTVHD, A259_Y262delinsEEF, S264_L267delinsDQTQ, Q269L, Y275F, M280T, and K282insFHR.

In some embodiments, the building block substitutions to D1 are selected from human DNASE1-LIKE 3 Isoform 1 (D1L3) and result in variants of human D1, which feature one or more of the following mutations: M1_L6delinsMSRE, G8_A9deinsAP, A12L, A14_G19delinsLLSIHS, V21_K24delinsLAMR, A26_A27delinsCS, I30_T32delinsVRS, S36T, M38_Y46delinsQEDKNAMDV, Q49_S52delinsKVIK, Y54C, A57I, Q60M, V62_R63delinsIK, H66_K72delinsNNRICPI, L74_N76delinsMEK, Q79_D83delinsRNSRRGI, H86N, V89I, E91_P92delinsSR, S97T, R101Q, L103A, V105L, R107_Q110delinsKEKL, A113_D115delinsVKR, Y118H, D120H, G122_N128delinsYQDGDA, T130V, N132S; A136_I137delinsFV, R139W, F141Q, R143_F144delinsPH, E146A, R148_E149delinsKD, A151V, V153I, A157_A158delinsTT, G160_A162delinsETS, A164K, A168E, Y170 D171delinsVE, L174T, Q177_K179delinsKHR, G181_L182delinsKA, D184_L187delisNFIF, R199_Q202delisPKKA, S204_S205delinsKN, W209R, S211D, T213R, Q215V, P219G, S221_A222delinsQE, A226_P228delinsVKKS, H230N, V238_A239delinsLR, M241_A246delinsQEIVSS, D250K, A252_P254delinsNSV, N256D, A259K, G262K, S264_L267delinsTEEE, Q269_I271delinsLDV, Y275F, V279_M280delinsFK, and K282delinsQSSRAFTNSKKSVTLRKKTKSKRS.

In some embodiments, the building block substitutions to D1 are selected from human DNASE1-LIKE 3 Isoform 2(D1L3-2) and result in variants of human D1, which feature one or more of the following mutations: M1_L6delinsMSRE, G8_A9deinsAP, A12L, A14_G19delinsLLSIHS, V21_K24delinsLAMR, A26_A27delinsCS, I30_T32delinsVRS, T36S, M38_Y46delinsQEDKNAMDV, Q49_S52delinsKVIK, Y52C, A57I, Q60M, V62_R63delinsIK, H66_K72delinsNNRICPI, L74_N76delinsMEK, Q79_Y106del, P108_Q110delinsEKL, A113_D115delinsVKR, Y118H, D120H, G122_N128delinsYQDGDA, T130V, N132S, A136_I137delinsFV, R139W, F141Q, R143_F144delinsPH, E146A, R148_E149delinsKD, A151V, V153I, A157_A158delinsAA, G160_A162delinsETS, A164K, Y170_D171delinsVE, L174T, Q177_K179delinsKHR, G181_L182delinsKA, D184_L187delisNFIF, R199_Q202delisPKKA, S204_S205delinsKN, W209R, S211D, T231R, Q215V, P219G, S221_S222delinsQE, A226_P228delinsVKKS, H230N, V238_A239delinsLR, M241_A246delinsQEIVSS, D250K, A252_P254delinsNSV, N256D, G262K, S264_L267delinsTEEE, Q269_I271delinsLDV, Y275F, V279_M280delinsFK, and K282delinsQSSRAFTNSKKSVTLRKKTKSKRS.

In various embodiments, the D1 variant evaluated in accordance with the disclosure comprises the D1 wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)₃.

In various embodiments, the peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In various embodiments, the human DNASE1-LIKE 1 (D1L1) variant evaluated and selected for use in therapy in accordance with embodiments of the invention comprise an amino acid sequence that is at least 80% identical to the enzyme defined by SEQ ID NO: 2, with one or more building block substitutions.

In some embodiments, the building block substitutions to D1L1 are selected from non-human D1L1 proteins and result in variants of human D1L1 which feature one or more of the following mutations: A26T, Q27H, A32T, A32S, V34L, A35T, A35I, R36K, Q38S, Q38E, Q38H, Q38Y, Q38P, Q38D, M40K, M40L, T42I, L43F, R45Q, R45K, L47V, M53T, S61A, S62T, G63Q, G63D, G63N, G63S, S64N, S64A, S64K, S64T, A65T, P66L, P66S, L68F, R71Q, R71E, E72K, N74S, R75K, F76Y, D77K, D77Q, D77Y, D77G, G78A, G78S, G78N, G78D, G80R, G80K, P81S, P81F, P81C, S83R, T84F, T84S, L85H, S86N, S86K, P88S, P88D, Q89L, Q89M, S93N, S93G, T94A, M96V, M96K, 198K, V100A, F102I, H106D, K107R, K107E, K107R, T108A, Q109E, V110L, L111R, S112N, S112D, S112E, S113F, V115Q, V115L, V115M, N117D, N117E, N117P, N177S, E119T, E119Q, E119K, V122I, V122L, A124T, A130G, A130C, Q131H, Q131W, S133T, L134F, P135R, N137D, N137K, V138T, V138I, L142V, V143A, A153D, K156P, K156N, K156T, L159K, Y162H, D163E, D163T, E167D, V168A, S169Y, S169A, Q170R, Q170G, H171R, S174N, S174T, K175E, K175Q, S176N, V177M, V177I, A188T, T191A, T191N, D196K, D196N, D196S, D196A, D196G, E199L, E199A, E199V, E203K, E203D, E203Q, P204A, P204V, P204T, H207R, H207S, V209A, 1210V, A211P, E214D, E241V, H223N, T225A, V229I, L231V, L231M, E234Q, E234V, R235G, R235T, R235L, C236L, R237Q, S238M, S238K, S238G, L240M, H241K, H241Q, H241S, H241R, T242A, T242S, T242N, T242G, A244T, D247N, T240K, T250R, Q250Q, S251T, S251R, Q252R, Q252G, T255N, T255S, E258Q, E259Q, N261R, N261K, M261T, 1262V, E271D, K273S, K273N, K273D, K273A, L274del, S275Q, S275K, S275R, Q276A, A277T, A277V, H278P, H278Q, S279G, S279N, S279R, C279S, 1280V, A280V, Q281P, Q281L, L283H, L283P, S284Y, S284C, S284H, S284G, T286A, T286S, T286V, V287T, V287A, F287F, G287V, L288A, L288S, L289S, L289V, L289M, S292L, S292P, S295P, S295T, S295A, P296S, Q297E, L298C, C299D, C299G, C299S, P300L, A301Q, A301V, and A302M.

In some embodiments, the building block substitutions to D1L1 are selected from human D1 and result in variants of human D1L1 which feature one or more of the following mutations: M1delinsMRGM, H2_T5delinsKLLG, F9A, A13_N14delinsLQ, Q17_R20delinsVLSK, C22A, A26I, R28_A32delinsTFGET, V34_L43delinsMSNATLVSYI, R45Q, A48S, C50Y, M53_L55delinsALV, V59R, S62_S64delisHLT, I66_L68delinsVGK, R71_E72delinsDN, R75_F76delinsQ, G78_P81delinsAPDT, S83_S86delinsHYVV, P88_Q89delinsEP, S93_T94delinsNS, M96K, T98R, V100_F102delinsLFV, S105_S112delinsPDQVSAVD, V115Y, N117D, E119_D120delinsGCEPCGN, V122T, F128_Q131delinsAIVR, S133_L144delinsFSRFTEVREFAI, T149_T150delinsAA, K152_K156delinsGDAVA, L158_N159delinsID, F165Y, E167D, Q173_K175delinsGLE, M188L, D186G, A188_E199delinsSYVRPSQWSSIR, R201W, E203S, G205T, H207Q, V209L, A211P, G213_E214delinsSA, V218_S221delinsATP, T225A, V229I, L231_H232delinsVA, E234_C236delinsMLL, S238_T242delinsGAVVPDS, A244_A245delinsLP, D247N, P249_Q253delinsQAAYG, T255_E258delinsSDQL, L260_N261delinsQA, E271M, and L274_A302del.

In some embodiments, the building block substitutions to D1L1 are selected from human D1L2 and result in variants of human D1L1 which feature one or more of the following mutations: H2_Y3delinsGG, T5R, F9_L12delinsWALE, N14A, A16_Q17delinsTA, F19L, C22G, A26I, R28_A32delinsSFGDS, A35_R45delinsSDPACGSIIAK, R49_50CdelinsGY, I52_L55delinsLALV, V59R, S61_G63delinsPDL, I66_L68delinsVSA, L70_L73delinsMEQI, R75_P81delinsSVSEHE, T84_L85delinsFV, P88_Q89delinsQP, S93_194delinsDQ, M96K, 198M, V100_F102delinsLFV, S105_Q109delinsKDAVS, L111_S113delinsVDT, V115L, Ni17P, E119_PI20delinsPE, A124S, A130_Q131delinsVK, L134A, S136_V138delinsGTGERAPP, S141_L142insRRALTPPPLPAAAQN, V145I, T149_T150delinsAA, K152_K156delinsHQAVA, L158_N159delinsID, F165Y, E167D, S169_H171delinsIDK, V177_L179delinsMLF, A188_E199delinsSYVRAQDWAAIR, T202_G205delinsSSEV, H207K, V209L, A211P, G213_E214delinsSA, R219_H223delinsGNSD, T225A, V229I, L231_H232delinsAC, E234A, C236L, S238_L239delinsRS, H241_T242delinsKPQS, A244_F246delinsTVH, P249_S251delinsQEE, Q253G, T255_E258delinsDQTQ, N261A, Y266F, E271T, and L274_A302delinsFHR.

In some embodiments, the building block substitutions to D1L1 are selected from human DNASE1-LIKE 3 Isoform 1 (D1L3) and result invariants of human D1L1, which feature one or more of the following mutations: H2_T5delinsSREL, L7_L8insPLL, F9L, I11_G14delinsLSIHS, Q17L, F19M, A23S, A26_A32delinsVRSFGES, V34_V39delinsQEDKNA, T42_L43delinsVI, R45_A48delinsKVIK, M53_Q56delinsILVM, V58_V59delinsIK, S62_I66delinsNNRIC, L68I, L70_E72delinsMEK, F76_S79delinsNSRR, G80_P81delinsIT, S83_S86delinsNYVI, P88_Q89delinsPQ, S92N, M96K, T98Q, V100_F102delinsAFL, R104_Q109delinsKEKLVS, L11_S112delinsKR, V115H, N117H, E119_D120delinsYQDGDA, A124S, A130_Q131delinsVW, S133_L134delinsQS, S136_L142delinsHTAVKDF, L144_V145delinsII, K152_E155delinsETSV, L158_A160delinsIDE, Y162_D163delinsVE, F165_E167delinsVTD, S169_H171delinsKHR, Q173_V177delinsKAENF, D186G, A188_T191delinsSYVP, R194_E199delinsAWKNIR, E203D, G205R, H207V, V209L, A211G, G213Q, R219_A220delinsKK, H223N, T225A, V229I, H232R, E234_R237delinsQEIV, L239_A245delinsSVVPKSNSV, P249_Q253delinsQKAYK, N261_I262delinsDV, Y266F, V270_E271delinsFK, K273_L274delinsQS, Q276R, H278_L283delinsFTNSKK, L285V, and V287_A302delinsLRKKTKSKRS.

In some embodiments, the building block substitutions to D1L1 are selected from human DNASE1-LIKE 3 Isoform 2 (D1L3-2) and result in variants of human D1L1, which feature one or more of the following mutations: H2_T5delinsSREL, A7delinsPLL, F9L, I11_G14delinsLSIHS, Q17L, F19M, S23A, A26_A32delinsVRSFGES, V34_V39delinsQEDKNA, T42_L43delinsVI, R45_A48delinsKVIK, M53_Q56delinsILVM, V58_V59delinsIK, S62_I66delinsNNRIC, L68I, L70_E72delinsMEK, F75_Q103del, S105_Q109delinsEKLVS, L111_S112delinsKR, V115H, Ni17H, E119_D120delinsYQDGDA, A124S, A130_Q131delinsVW, S133_L134delinsQS, S136_L142delinsHTAVKDF, L144_V145delinsII, K152_E155delinsETSV, L158_A160delinsIDE, Y162_D163delinsVE, F165_E167delinsVTD, S169_H171delinsKHR, Q173_V177delinsKAENF, D186G, A188_T191delinsSYVP, R194_E199delinsAWKNIR, E203D, G205R, H207V, V209L, A211G, G213Q, R219_A220delinsKK, H223N, T225A, V229I, H232R, E234_R237delinsQEIV, L239_A245delinsSVVPKSNSV, P249_Q253delinsQKAYK, N261_I262delinsDV, Y266F, V270_E271delinsFK, K273_L274delinsQS, Q276R, H278_L283delinsFTNSKK, L285V, and V287_A302delinsLRKKTKSKRS.

In certain embodiments, the D1L1 protein variant contains one or more amino acid substitutions, additions, or deletions in the C-terminal tail domain defined by SEQ ID NO: 8. The C-terminal tail domain or a portion thereof may be deleted. In some embodiments, at least 3, 5, 8, 10, 12, 15, 18, or 23 amino acids of the C-terminal tail domain are deleted.

In various embodiments, the D1L1 variant evaluated in accordance with the disclosure comprises the D1L1 wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence, which is also herein referred to as a peptide linker, or a linker, can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)₃.

The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In various embodiments, the human DNASE1-LIKE 1 (D1L2) variant evaluated and selected for therapy in accordance with this disclosure comprises an amino acid sequence that is at least 80% identical to the enzyme defined by SEQ ID NO: 3, with one or more building block substitutions.

In some embodiments, the building block substitutions to D1L2 are selected from non-human D1L2 proteins and result in variants of human D1L2, which feature one or more of the following mutations: L22K, I24V, I29V, S35N, S35H, S35R, S35T, V37A, S38L, A41D, A41V, A41G, G43I, S44G, S44i, I45V, K48Q, L55I, L55V, A56T, A56M, P64A, S70D, S70T, A71T, A71L, A71S, A71V, M73L, E74Q, N77H, S78R, E81K, E81R, E83N, S85G, S85N, Q90E, Q90K, Q96H, F103Y, V104I, K107D, A109V, A109T, A109K, V110A, V113L, V113M, D114S, D114E, L117Q, P119S, E122G, V124A, V124F, S126N, E128D, F134V, A136V, A136T, G138S, G138R, T139S, T139C, S148C, A151P, P154A, A159P, A160G, A161P, A161T, Q162D, Q162K, Q162R, Q162T, N163K, N163E, L164V, L164F, 1167V, H174N, Q175H, A178T, A178V, D192N, G195N, T196S, D198V, M199L, M199I, S210K, R213K, Q215H, A218P, A219S, E226Q, V227I, S243T, A252V, C253S, A255S, A255V, R256H, L257M, R259K, S260T, L261V, Q264H, T267S, T267A, D270N, G276D, G276S, T280S, T280D, T280A, A284C, I286V, L295F, F297S, F297T, F297P, H298R, and R299del.

In some embodiments, the building block substitutions to D1L2 are selected from human D1 and result in variants of human D1L2, which feature one or more of the following mutations: G2R, P4_A6delinsMK, A9G, W12L, E15A, A16_G18delinsLLQ, T19_A21delinsAVS, R23K, G25A, S31T, D34_S35delinsET, V37M, D39_G43delinsNATLV, I45Y, A47_K48delinsVQ, A51_G52delinsSR, L55I, P64_D65delinsSH, S67T, S70_A71delinsGK, M73_I76delinsLDNL, S78_E83delinsQDAPDT, S85_F86delinsHY, S88V, Q90E, D95_Q96delinsNS, M100R, K107P, A109Q, V112A, T115S, L117Y, P119D, P121_E122delinsGCEPCGN, V124T, S126N, F130_V131delinsI, K133R, S135_G138delinsFSRF, G140_I167delinsEVREFAIV, H174_Q175delinsGD, I191_D192delinsQE, T196_D197delinsLE, M199_L202delinsVMLM, D208G, A214_D216delinsPSQ, A218_A219delinsSS, R223_S224delinsWT, E226_V227delinsPT, K229Q, V240_D244delinsATPTH, A252_C253delinsVA, A225_R256delinsML, R259_K262delinsGAVV, Q264D, 1267_D270delinsLPFN, E273_E275delinsAAY, D278_Q281delinsSDQL, L283Q, F283Y, T294M, and F297_R299del.

In some embodiments, the building block substitutions to D1L2 are selected from human D1L1 and result in variants of human D1L2, which feature one or more of the following mutations: G2_G3delinsHY, R5T, W12_E15delinsFLIL, A17N, T19_A20delinsAQ, L22F, G25C, I29A, S31_S35delinsRLTLA, S38_K48delinsAREQVMDTLVR, G52_Y53delinsRC, L55_V58delinsIMVL, R62V, P64_L66delinsSSG, V69_A71delinsIPL, M73_I76delinsLREL, S78_E83delinsRFDGSGP, F86_V87delinsTL, Q90_P91delinsPQ, D95_Q96delinsST, K98M, M100T, L102_V104delinsVYF, K107_S111delinsSHKTQ, V113_T115delinsLSS, L117V, P119N, P121_E122delinsED, S126A, V132_K133delinsAQ, A136L, G138_P145delinsSNV, R149_N163del, I167V, A171_A172delinsTT, H174_A178delinsKAVEK, I180_D181delinsLN, Y187F, D189E, I191_K193delinsSQH, M199_F201delinsVIL, S210_R221delinsASLTKKRLDKLE, S224_V227delinsTEPG, K229H, L231V, P233A, S235_A235delinsGE, G241_D244delinsRASTH, A246T, 1250V, A252_C253delinsLH, A255E, L257C, R259_S260delinsSL, K262_S265delinsHT, T267_H269delinsAAF, Q272_E274delinsPTS, G276Q, D278_Q281delinsTEEE, A284N, F289Y, T294E, and F297_R299delinsLSQAHSVQPLSTVLLLLSLLSPQLCPAA.

In some embodiments, the building block substitutions to D1L2 are selected from human DNASE1-LIKE 3 Isoform 1 (D1L3) and result in variants of human D1L2, which feature one or more of the following mutations: G2_R5delinsSREL, L7P, A9_A10delinsL, W12_A13delinsLL, S15_A20delinsSIHSAL, L22M, G25_A26delinsCS, I29_Q30delinsVR, D34E, V37_S38delinsQE, P40_I45delinsKNAMDV, A47V, I49_Y53delinsVIKRS, L55_A56delinsII, Q59M, V61_R62delinsIK, P64_A71delinsSNNRICPI, Q75_I76delinsKL, N77_S78insRS, V79_E83delinsRRGIT, S85_F86delinsNY, S88I, Q90_P91delinsSR, D95_Q96delinsNT, M100Q, L102A, V104L, R106_A109delinsKEKL, V113_T115delinsKRS, L117H, P119H, P121_E122delinsYQDGDA, K133W, S135_A136delinsQS, G138H, G140_A159del, A161_L164delinsVKDF, L166I, A171_A172delinsTT, H174_A176delinsETS, A178K, A182E, Y184 D185delinsVE, L188T, I191_K193delinsKHR, G195_L200delinsKAENFI, L202M, D208G, R213_D126delinsPKKA, A218_A129delinsKN, S224_V227delinsTDPR, K229V, P233G, S235_A236delinsQE, G241_N242delinsKK, S243_D244insTN, A252_C253delinsLR, A255_R259delinsQEIVS, L261_K262delinsVV, Q264K, A266_T267delinsNS, H269F, E273_G276delinsKAYK, D278_Q281delinsTEEE, A284_I285delinDV, V293_T294delinsFK, K296_H298delinsQSS, and R295insAFTNSKKSVTLRKKTKSKRS.

In some embodiments, the building block substitutions to D1L2 are selected from human DNASE1-LIKE 3 Isoform 2 (D1L3-2) and result in variants of human D1L2, which feature one or more of the following mutations: G2_R5delinsSREL, L7P, A9_A10delinsL, W12_A13delinsLL, S15_A20delinsSIHSAL, L22M, G25_A26delinsCS, I29_Q30delinsVR, D34E, V37_S38delinsQE, P40_I45delinsKNAMDV, A47V, I49_Y53delinsVIKRS, L55_A56delinsII, Q59M, V61_R62delinsIK, P64_A71delinsSNNRICPI, Q75_I76delinsKL, K107_A109delinsEKL, V113_T115delinsKRS, L117H, P119H, P121_E122delinsYQDGDA, K133W, S135_A136delinsQS, G138H, G140_A159del, A161_L164delinsVKDF, L166I, A171_A172delinsTT, H174_A176delinsETS, A178K, A182E, Y184_D185delinsVE, L188T, I191_K193delinsKHR, G195_L200delinsKAENFI, L202M, D208G, R213_D126delinsPKKA, A218_A129delinsKN, S224_V227delinsTDPR, K229V, P233G, S235_A236delinsQE, G241_N242delinsKK, S243_D244insTN, A252_C253delinsLR, A255_R259delinsQEIVS, L261_K262delinsVV, Q264K, A266_T267delinsNS, H269F, E273_G276delinsKAYK, D278_Q281delinsTEEE, A284_I285delinDV, V293_T294delinsFK, K296_H298delinsQSS, and R295insAFTNSKKSVTLRKKTKSKRS.

In certain embodiments, the D1L2 protein variant contains one or more amino acid substitutions, additions, or deletions in the proline-rich extension domain defined by SEQ ID NO: 9. The proline-rich extension domain or a portion thereof may be deleted, including a deletion (or truncation) of at least 3 amino acids, at least 5 amino acids, or at least 10 amino acids.

In various embodiments, the D1L2 variant evaluated in accordance with the disclosure comprises the D1L2 wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)3.

The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In some embodiments, the human DNASE1-LIKE 3 Isoform 1 (D1L3) variant evaluated and selected for use in therapy in accordance with embodiments of the invention comprise an amino acid sequence that is at least 80% identical to the enzyme defined by SEQ ID NO: 4, with one or more building block substitutions.

In some embodiments, the building block substitutions to D1L3 are selected from non-human D1L3 proteins and result in variants of human D1L3 which feature one or more of the following mutations: M21L, K22R, I22L, I22V, E33A, E33Y, E33G, S34A, S34T, Q36K, Q36R, E37A, E37Q, D48N, K39Q, K39H, K39R, K39C, N40E, N40Q, N40K, A41V, V44I, V53I, I53L, I54M, V57L, I60V, N64S, H64S, R66N, R66M, I70V, I70M, I70T, M72L, E73K, K74R, R77G, R81K, G82S, I83V, I83T, T84M, T84K, S91P, T91V, T91A, L105V, K107M, V111L, S112T, R115T, R115A, R115K, R115D, R115Q, S116K, S116N, S116Y, H118L, H118V, Y119F, H120G, Y122N, Q123E, D124A, D124S, D124N, G125E, A127V, A127T, V129A, F135Y, V137T, Q140H, S141A, H143F, H143Y, V146A, I152V, T157S, T160A, V162I, K163R, V169A, E170D, T173M, T173L, V175M, K176R, K176Q, H177S, H177R, R178Q, K180E, K180N, K181T, K181V, E183A, E183Q, A201S, K203Q, K203R, R212K, R212N, R212G, R212M, V214I, G218K, G218A, Q220E, Q220D, K227R, K227S, K227E, N239K, N239S, N239H, R239C, Q241P, E242D, E242N, V244I, S245N, S245R, K250R, K250D, K250R, K250G, K250N, K250Q, N252S, S253G, S253L, V254T, V254I, D256N, Q258R, Y261F, K262D, K262E, K262L, K262R, K262Q, T264S, E266S, E267K, E267Q, E267K, D270N, D270E, V271I, S282E, R285T, F287I, S290N, K291R, V294I, T295S, T295Q, L296V, L296P, L296S, R297K, K299R, T300K, T300A, S302G, S302A, S302V, S302T, K303N, K303S, K303R, R304H, R304S, S305P, S305T, and S305A.

In some embodiments, the building block substitutions to D1L3 are selected from human D1 and result in variants of human D1L3 which feature one or more of the following mutations: M1_E4delinsMRGMKL, A6_P7delinsGA, L10A, L12_S17delinsAALLQG, L19_R22delinsVSLK, C24_S25delinsAA, V28_S30delinsIQT, S34T, Q36_V44delinsMSNATLVSY, K47_K50delinsQILS, C52Y, I55A, M58Q, I60_K61delinsVR, N64_I70delinsHLTAVGK, M72_K74delinsLDN, R77_I83delinsQDAPD, N86H, I89V, S91_R92delinsEP, T97S, Q101R, A103L, L105V, K107_L110delinsRPDQ, V113_R115delinsAVD, H118Y, H120D, Y122_A127delinsGCEPCGN, V129T, S131N, F135_V136delinsAI, W138R, Q140F, P142_H143delinsRF, A145E, K147_D148delinsRE, V150A, I152V, T156 T157delinsAA, E159 S161delinsGDA, K163A, E167A, V169_E170delinsYD, T173L, K176_R178delinsQEK, K180_A181delinsGL, N183_F186delinsDVML, P198_A201delinsRPSQ, K203_N204delinsSS, R208W, D210S, R212T, V214Q, G218P, Q220_E221delinsSA, V225_S228delinsATP, N230H, L238_R239delinsVA, Q241_S246delinsMLLRGA, K250D, N252_V254delinsALP, D256N, K259A, K262G, T264_E267delinsSDQL, L269_V271delinsQAI, F275Y, F279_K280delinsVM, and Q282_S205delinsK.

In some embodiments, the building block substitutions to D1L3 are selected from human D1L1 and result in variants of human D1L3 which feature one or more of the following mutations: S2_L5delinsHYPT, P7_L9delinsL, L11F, L13_S17delinsILANG, L19delinsQ, M21F, S25A, V28_S34delinsAQRLTLA, Q36_A41delinsVAREQV, V44_I45delinsTL, K47_K50delinsRILA, I55_M58delinsMVLQ, I60_K61delinsVV, N6_C68delinsSGSAI, I70L, M72_L74delinsLRE, N78 R81delinsFDGS, I83_T84delinsP, N86_I89delinsSTLS, S91_R92delinsPQ, N96S, K99M, Q101T, A103_L105delinsVYF, K107_S112delinsRSHKTQ, K114_R115delinsLS, H118V, H120N, Y122_A127delinsED, S131A, V137_W138delinsAQ, Q140_S141delinsSL, H143_F149delinsSNVLPSL, I151_I152delinsLV, E159_V162delinsKAVE, I165_A167delinsLNA, V169_E170delinsYD, Y172_D174delinsFLE, K176_R178delinsSQH, K180_F184delinsQSKDV, G193D, S195_P198delinsASLT, A201_R206delinsRLDKLE, D210E, R212G, V214H, L216V, G218A, Q220G, K226_K227delinsRA, N230H, A232T, I236V, R239H, Q246_V249delinsERCR, S246_V254delinsLLHTAAA, Q258_K262delinsPTSFQ, D270_V271delinsNI, F275Y, F279_K280delinsVE, Q282_S283delinsKL, R285Q, F287_K292delinsHSVQPL, V294L, and L296_S205delinsVLLLLSLLSPQLCPAA.

In some embodiments, the building block substitutions to D1L3 are selected from human D1L2 and result in variants of human D1L3 which feature one or more of the following mutations: S2_L5delinsGGPR, P7L, L9delinsAA, L11_L12delinsWA, S14_L19delinsEAAGTA, M21L, C24_S25delinsGA, V28_R29delinsIQ, E33D, Q36_E37delinsVS, K39_V44delinsPACGSI, A47V, V48_C52delinsILAGY, I54_I55delinsLA, M58Q, I60_K61delinsVR, S63_I70delinsPDLSAVSA, K74_L75delinsQI, XX (deletion in Donor), R80_T84delinsVSEHE, N86_Y87delinsSF, I89S, S91_R92delinsQP, N96_T97delinsDQ, Q101M, A103L, L105V, K107_L110delinsRKDA, K114_S116delinsVDT, H118L, H120P, Y122_A127delinsPE, W138K, Q140_S141delinsSA, H143G, T144_A145insGERAPPLPSRRALTPPPLPA, V146_F149delinsAQNL, I151L, T156_T157delinsAA, E159_S161delinsHQA, K163A, E162A, V169_E170delinsYD, T173L, K176_R178delinsIDK, K180_I185delinsGTDDML, M187L, G193D, P198_A201delinsRAQD, K203_N204delinsAA, T209_R212delinsSSEV, V214K, G218P, Q220_E221delinsSA, K226_K227delinsGN, T229_N230delinsD, L238_R239delinsAC, Q241_S245delinsARLRR, V247_V248delinsLK, K250Q, N252_S253delinsAT, F255H, K259_K262delinsEEFG, T264_E267delinsDQTQ, D270_V271delinsAI, F279_K280delinsVT, Q282_S284delinsKFH, and A286_S305del.

In certain embodiments, the D1L3 protein variant contains one or more amino acid substitutions, additions, or deletions in the C-terminal tail amino acid sequence defined by SEQ ID NO: 10. The C-terminal tail domain or a portion thereof may be deleted. In some embodiments, at least 3, 5, 8, 10, 12, 15, 18, or 23 amino acids of the C-terminal tail domain are deleted.

In certain embodiments, the D1L3 protein variant contains one or more, e.g., 1, 2, 3, 4, 5, or more amino acid substitutions, additions, or deletions in the internal sequence defined by SEQ ID NO: 11 (which is absent from isoform 2), and which is optionally deleted in whole or in part.

In various embodiments, the D1L3 variant evaluated in accordance with the disclosure comprises the D1L3 wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)₃.

The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris. Further, longer linker sequences showed improved chromatin-degrading activity, as compared to shorter linker sequences.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In some embodiments, the DNASE1-LIKE 3 Isoform 2 (D1L3-2) variant evaluated and selected for use in therapy in accordance with embodiments of the invention comprise an amino acid sequence that is at least 80% identical to the enzyme defined by SEQ ID NO: 5, with one or more building block substitutions.

In some embodiments, the building block substitutions to D1L3-2 are selected from non-human D1L3 proteins and result in variants of human D1L3-2 which feature one or more of the following mutations: M21L, K22R, I22L, I22V, E33A, E33Y, E33G, S34A, S34T, Q36K, Q36R, E37A, E37Q, D48N, K39Q, K39H, K39R, K39C, N40E, N40Q, N40K, A41V, V44I, V53I, I53L, I54M, V57L, I60V, N64S, H64S, R66N, R66M, I70V, I70M, I70T, M72L, E73K, K74R, R77G, V81L, S82T, R85T, R85A, R85K, R85D, R85Q, S86K, S86N, S86Y, H88L, H88V, Y89F, H90G, Y92N, Q93E, D94A, D94S, D94N, G95E, A97V, A97T, V99A, F105Y, V107T, Q110H, S111A, H113F, H113Y, V116A, I122V, T127S, T130A, V132I, K133R, V139A, E140D, T143M, T143L, V145M, K146R, K146Q, H147S, H147R, R148Q, K150E, K150N, K151T, K151V, E153A, E153Q, A171S, K173Q, K173R, R182K, R182N, R182G, R182M, V184I, G188K, G188A, Q190E, Q190D, K197R, K197S, K197E, N209K, N209S, N209H, R209C, Q211P, E212D, E212N, V214I, S215N, S215R, K220R, K220D, K220R, K220G, K220N, K220Q, N222S, S223G, S223L, V224T, V224I, D226N, Q228R, Y231F, K232D, K232E, K232L, K232R, K232Q, T234S, E236S, E237K, E237Q, E237K, D240N, D240E, V241I, S252E, R255T, F257I, S260N, K261R, V264I, T265S, T265Q, L266V, L266P, L266S, R267K, K269R, T270K, T270A, S272G, S272A, S272V, S272T, K273N, K273S, K273R, R274H, R274S, S275P, S275T, and S275A.

In some embodiments, the building block substitutions to D1L3-2 are selected from human D1 and result in variants of human D1L3-2 which feature one or more of the following mutations: M1_E4delinsMRGMKLL, A6_P7delinsGA, L10A, L12_S17delinsAALLQG, L19_R22delinsVSLK, C24_S25delinsAA, V28_S30delinsIQT, S34T, Q36_V44delinsMSNATLVSY, K152_K156delinsGDAVA, C52Y, I55A, M58Q, I60_K61delinsVR, N64_I70delinsHLTAVGK, M72_K74delinsLDN, N76_R77insQDAPDTYHYVVSEPLGRNSYKERYLFVY, E78_L80delinsPDQ, V83_R85delinsAVD, H88Y, H90D, Y92_A97delinsGCEPCGN, V99T, S101N, F105_V106delinsAI, W108R, Q110F, P112_H113delinsRF, A115E, K117_D118delinsRE, V120A, I122V, T126_T127delinsAA, E129_T131delinsGDA, K133A, V139_E140delinsYD, T143L, K146_R148delinsQEK, K150_A151delinsGL, N153_F156delinsDVML, P168_A171delinsRPSQ, K173_Ni174delinsSS, R178W, D180S, R182T, V184Q, G188P, Q190_E191delinsSA, V195_S199delinsATP, N200H, L208_R209delinsVA, Q211_S216delinsMLLRGA, K220D, N222_V224delinsALP, D226N, K232G, T234_E237delinsSDQL, L239_V241delinsQAI, F245Y, F249_K250delinsVM, and Q252_S275delinsK.

In some embodiments, the building block substitutions to D1L3-2 are selected from human D1L1 and result in variants of human D1L3-2 which feature one or more of the following mutations: S2_L5delinsHYPT, P7_L9delinsA, L11F, L13_S17delinsILANG, L19delinsQ, M21F, S25A, V28_S34delinsAQRLTLA, Q36_A41delinsVAREQV, V44_I45delinsTL, K47_K50delinsRILA, I55_M58delinsMVLQ, I60_K61delinsVV, N6_C68delinsSGSAI, I70L, M72_L74delinsLRE, N76_R77insRFDGSGPYSTLSSPQLGRSTYMETYVYFYRSHKTQ, E78_S82delinsSHKTQ, K84_R85delinsLS, H88V, H90N, Y92_A97delinsED, S101A, V107_W108delinsAQ, Q110_S111delinsSL, H113_F119delinsSNVLPSL, I121_I122delinsLV, E129_V132delinsKAVE, I135_A137delinsLNA, V139_E140delinsYD, Y142_D144delinsFLE, K146_R148delinsSQH, K150_F154delinsQSKDV, G163D, S165_P168delinsASLT, A171_R176delinsRLDKLE, D180E, R182G, V184H, L186V, G188A, Q190G, K196_K227delinsRA, N200H, A192T, I206V, R209H, Q216_V219delinsERCR, S216_V224delinsLLHTAAA, Q228_K232delinsPTSFQ, D240_V241delinsNI, F245Y, F249_K250delinsVE, Q252_S253delinsKL, R255Q, F257_K262delinsHSVQPL, V264L, and L266_S275delinsVLLLLSLLSPQLCPAA.

In some embodiments, the building block substitutions to D1L3-2 are selected from human D1L2 and result in variants of human D1L3-2 which feature one or more of the following mutations: S2_L5delinsGGPR, P7L, L9delinsAA, LL11_L12delinsWA, S14_L19delinsEAAGTA, M21L, C24_S25delinsGA, V28_R29delinsIQ, E33D, Q36_E37delinsVS, K39_V44delinsPACGSI, A47V, V48_C52delinsILAGY, I54_I55delinsLA, M58Q, I60_K61delinsVR, S63_I70delinsPDLSAVSA, K74_L75delinsQI, E78_L80delinsKDA, K84_S86delinsVDT, H88L, H90P, Y92_A97delinsPE, W108K, Q110_S111delinsSA, H113G, T114_A115insGERAPPLPSRRALTPPPLPA, V116_F119delinsAQNL, I121L, T126_T127delinsAA, E129_S141delinsHQA, K133A, E132A, V139_E140delinsYD, T143L, K146_R148delinsIDK, K150_I155delinsGTDDML, M157L, G163D, P168_A171delinsRAQD, K173_N174delinsAA, T179_R182delinsSSEV, V184K, G188P, Q190_E191delinsSA, K96_K197delinsGN, T199_N200delinsD, L208_R209delinsAC, Q211_S215delinsARLRR, V217_V218delinsLK, K220Q, N222_S223delinsAT, F225H, K229_K242delinsEEFG, T234_E237delinsDQTQ, D240_V241delinsAI, F249_K250delinsVT, and Q252_S254delinsKFH, A256_S275del.

In certain embodiments, the D1L3-2 protein variant contains one or more amino acid substitutions, additions, or deletions in the C-terminal tail domain defined by SEQ ID NO: 11. The C-terminal tail domain or a portion thereof may be deleted. In some embodiments, at least 3, 5, 8, 10, 12, 15, 18, or 23 amino acids of the C-terminal tail domain are deleted.

In various embodiments, the D1L3-2 variant evaluated in accordance with the disclosure comprises the D1L3-2 wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)₃.

The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris, and may provide for improved chromatin degrading activity.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In various embodiments, the DNASE2A (D2A) variant evaluated and selected for use in therapy in accordance with embodiments of the invention comprise an amino acid sequence that is at least 80% identical to the enzyme defined by SEQ ID NO: 6, with one or more building block substitutions.

In some embodiments, the building block substitutions to DNASE2A are selected from non-human D2A proteins and result in variants of human D2A, which feature one or more of the following mutations: Q25R, L38H, L38N, R39S, R39T, G40S, G42R, E43D, A44T, A44K, A44V, A45P, A45T, R47K, R47N, R47S, Q50T, Q50M, Q50R, L54M, L54F, E56Q, S57N, S57H, S57E, G59D, G59E, G60D, R62Q, R62S, R65V, R65A, A66G, L67Y, L67H, L67F, L67S, N69D, P71S, P71K, P71T, E72D, E72T, V75L, R77L, Q80L, R84Q, S85K, S85N, T87S, T87N, L93V, Q101K, P102S, P102Y, S103R, K104S, K104E, K104G, A105S, Q106R, Q106K, D107H, S109T, M110G, M110S, M110N, R111H, H122Q, D123E, V129I, N134R, P137S, P138R, A139S, A142G, A143V, S145T, H148P, S149N, S149G, C151Q, C151R, T152K, Y153F, L 158I, F162L, F164L, A165T, A165S, S168A, S168P, S168L, K169R, K169G, K169D, K169N, M170I, G171S, K172R, W180L, W180M, N183D, Y184H, Q185K, Q185R, I180F, I180D, Q192R, E193K, F194L, D196Y, N199T, N199E, V201I, V201T, G203N, G203Q, S207L, S207R, Q208H, Q208R, E209G, I215V, T216I, Q220R, Q220K, A221K, A223T, V224T, V224S, F231C, S232G, K233N, A244S, A245E, T249S, N250T, H257Q, H257P, T259S, V260P, V260S, V260A, D269G, I270A, I270T, I270V, W271Y, W271H, W271Q, Q272K, Q272H, V273I, L274F, N275D, N277T, Q278E, I279T, A280G, A285S, G286R, P287L, S288T, S288A, S288N, N290S, S291A, S301A, S301T, K303Q, K303E, G304R, T307A, T307V, Q316K, G317A, G317R, E319T, Q320H, L326V, A328T, L330V, L330M, A332S, L333F, Q338R, Q338K, P339S, N343D, N343A, Y344W, Y344C, Q345K, and Q345E.

In some embodiments, the building block substitutions to D2A are selected from human DNASE2B (D2B) and result in variants of human D2A, which feature one or more of the following mutations: I2_L6delinsKQKMM, A8R, C11_P13delinsRTSFALLFLGLFGVLG, G15_T18delinsATIS, Y20_S23delinsRNEE, Q25_P26delinsKA, V31_V32delinsTF, A37_L38delinsK, G40_G42delinsQNK, A44_R47delinsSGET, Q50E, K52L, E56_G60delinsSTTRS, D62_A66delinsKSEQ, I68M, S70_V75delinsDTKSVL, S78T, P81Q, R84delinsEAYA, N86_L90delinsKSNNT, F92Y, L94I, Q108_P109delinsGV, Q101K, S103_D107delinsVNK, V117L, L120_G124delinsWNRVQ, V129I, V132I, N134Q, P136del, A139_A143delinsIPEEG, S145_W146delinsDY, H148_Y153delinsPTGRRN, T156_L158delinsSGI, V160_S161delinsIT, P163_A165delinsKYN, F167_K172delinsYEAIDS, T175_Y178delinsLVCN, W180N, N183_I89delinsSCSIPAT, A192H, F194_V194delinsLIHMPQLCTRASS, Q208_E209delinsEI, W211_I215delinsGRLLT, T218Q, Q220_A221delnsAQ, A223_V224delinsQK, Q226_S227delinsLH, F231_K233delinsSDS, G235L, L238_G241delinsIFAA, L243M, A245_A246delinsQR, G248K, N250H, Q252_F255delinsLTET, H257_I262delinsQRKRQE, D269_Q272delinsLPYH, L274Y, Y276_Q278delinsIKA, A280_S288delinsKLSRHSY, N290S, T292_E293delinsYQ, S296A, V300I, P302Q, P305delinsTKNR, V309I, M312L, N315_Q320deinsSPHQAF, G322S, T325_L326delinsFI, L330_K335delinsNWQIYQ, P339G, K342_N343delinsLY, Q345_P346delinsES, and N348_I360delinsK.

In various embodiments, the D2A variant evaluated in accordance with the disclosure comprises the D2A wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)₃.

The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In some embodiments, the DNASE2B (D2B) variant evaluated and selected for therapy in accordance with embodiments of this disclosure comprise an amino acid sequence that is at least 80% identical to the enzyme defined by SEQ ID NO: 7, with one or more building block substitutions.

In some embodiments, the building block substitutions to D2B are selected from non-human D2B proteins and result in variants of human D2B, which feature one or more of the following mutations: A28P, A28T, T29E, T29V, T29K, S31A, R33I, N34S, E36Y, E36D, A37P, T44I, T44A, T44V, K50R, R51Q, R51K, Q52T, N53S, N53D, N53E, K54R, E55A, E55G, S56G, G57E, G57T, G57R, T59A, T59M, E62Q, E62D, E62G, T70R, T70M, T70I, R71Q, S72T, R74N, R74S, R74K, K75R, E77L, E77H, E77K, Q78Y, Q78H, Q78L, M80I, M80V, D82T, D82S, D82A, T83S, K84R, K84D, V86A, V86S, Q92E, Q93H, E96D, A97T, Y98H, Y98N, Y98C, A99D, A99H, S100A, S100F, K101E, S102T, S102N, S102D, N104D, N104S, L108V, I109L, G113A, V114I, K116G, K116A, P117S, V118A, N119T, N119G, N119S, Y120C, R122G, K123Q, K123N, Y124F, T127A, L132V, V136T, V136I, I145V, Q147K, Q147R, I151V, I151T, E154H, E154K, D157E, P160T, P160S, T161S, R164Q, N165Y, N165H, G166A, S168T, S168A, S168N, I170L, I170M, F174L, K175G, K175R, N178S, Y179F, A181E, A181T, S184F, V188I, C189L, C189F, C189Y, N190Q, V192I, S195R, S197F, A200S, A200N, A200T, T201I, T201A, H203R, Q204W, Q204M, E205K, I207V, I207F, H208Y, H208Y, M209L, Q211R, L212M, T214A, R215K, R215G, A216S, S217T, S217H, S218A, S219L, E220K, G223V, G223S, R224Q, L225Y, L225R, L225H, T227A, T228E, T228V, T228S, Q230H, Q233R, Q235L, K236N, K236S, L238V, L238I, S243F, D244S, D244T, S245F, F246Y, L247T, L247H, A252T, A252V, A253G, M255I, R258K, R258H, R258Q, T261V, T265A, T265V, E266Q, T267S, R270K, R272K, R272N, R272G, Q273H, Y283H, C285I, I288V, A290S, K292G, K292R, L293V, L293G, L293I, R295G, R295S, R295L, R295H, H296K, H296Q, Y298D, S300P, Y302R, Y302H, Q303H, A306S, I310V, Q312I, Q312T, Q312R, Q312L, G314D, G314R, T315S, K316A, K316Q, N317A, R318H, P329L, H330Y, F333L, F333S, S335G, T341S, T341N, Q342K, W344H, W344R, W344Q, Q345H, Q345Y, Q345R, Q345N, Q349H, Q351H, Q351D, Q351E, G352K, G352R, V354Y, L355S, Y356R, Y356H, Y357H, E358G, E358A, S359F, S359N, S359D, and K361N.

In others embodiments, the building block substitutions to D2B are selected from human DNASE2A (D2A) and result in variants of human D2B, which feature one or more of the following mutations: K2_M6delinsIPLLL, R8A, R11_G21delinsCVP, A28_S31delinsGALT, R33_E36delinsYGDS, K38_A39delinsQP, T44_F45delinsVV, K50delinsAL, Q52_K54delinsGSG, S56_T59delinsAAQR, E62Q, L64K, S70_S72delinsESSGG, K75_Q78delinsDGRA, M80I, D82_K87delinsSPEGAV, T90S, Q93P, E96_Y99delinsR, K101_T105delinsNTSQL, Y107F, I109L, G113_V114delinsQP, K106Q, V118_Y I20delinsSKAQD, L130V, W133_Q137delinsLDHDG, I142V, I145V, Q147N, F148_P149insP, I151 G155delinsASSAA, D157 Y158delinsSW, P160_N165delinsHSACTY, S168_I170delinsTLL, I172_T173delinsVS, K175_N177delinsPFA, Y179_s184delinsFSKMGK, L187_N190delinsTYTY, N192W, S195_T201delinsNYQLEGI, H203A, L206_S218delinsFPDLENVVKGHHV, E220_I221delinsQE, G223_T227delinsWNSSI, A232_Q233delinsQA, Q236_K236delinsAV, L238_H239delinsQS, S243_S2456delinsFSK, L247G, I250_A253delinsLYSG, M255L, Q257_R258delinsAA, K260G, H262N, L264_T267delinsQVQF, Q269_E274delinsHKTVGI, L281_Y284delinsDIWQ, Y286L, I288_A290delinsVNQ, K292_Y298delinsAFPGAGPS, S300N, Y302_Q303delinsTE, A306S, I310V, Q312P, T315_R318delinsP, I322V, L325M, S328_F333delinsNQGEEQ, S335G, F338_I339delinsTL, N343_Q348delinsLPALWK, G353P, L355_Y356delinsKN, E358_S359delinsQP, and K361delinsNGMARKPSRAYKI.

In various embodiments, the D2B variant evaluated in accordance with the disclosure comprises the D2B wildtype amino acid sequence or a variant sequence described herein, fused to the C-terminus of a carrier protein, with a linking amino acid sequence. In some embodiments, the carrier protein is Fc fragment or albumin. In some embodiments, the carrier protein is human albumin. The linking sequence can be a flexible linker predominately composed of Gly, or Gy and Ser. In some embodiments, the linker is composed of Gly and amino acids having hydrophilic side chains (e.g., Ser, Thr). In some embodiments, the linker is from 5 to 20 amino acids. An exemplary linker has the structure (GGGGS)₃.

The peptide linker may be a flexible linker, a rigid linker, or in some embodiments a physiologically-cleavable linker (e.g., a protease-cleavable linker). In some embodiments, the linker is 5 to 100 amino acids in length, or is 5 to 50 amino acids in length.

Linkers, where present, can be selected from flexible, rigid, and cleavable peptide linkers. Flexible linkers are predominately or entirely composed of small, non-polar or polar residues such as Gly, Ser and Thr. An exemplary flexible linker comprises (Gly_{y}Ser)ₙ linkers, where y is from 1 to 10 (e.g., from 1 to 5), and n is from 1 to about 10, and in some embodiments, is from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 2 to 6). Generally, linkers can be predominately composed of amino acids selected from Gly, Ser, Thr, Ala, and Pro. Exemplary linker sequences contain at least 10 amino acids, and may be in the range of 15 to 35 amino acids. Exemplary linker designs are provided as SEQ ID NOS: 41 to 51.

In some embodiments, the variant comprises a linker, wherein the amino acid sequence of the linker is predominately glycine and serine residues, or consists essentially of glycine and serine residues. In some embodiments, the ratio of Ser and Gly in the linker is, respectively, from about 1:1 to about 1:10, from about 1:2 to about 1:6, or about 1:4. Exemplary linker sequences comprise S(GGS)₄GSS (SEQ ID NO: 46), S(GGS)₉GS (SEQ ID NO: 47), (GGS)₉GS (SEQ ID NO: 48). In some embodiments, the linker has at least 10 amino acids, or at least 15 amino acids, or at least 20 amino acids, or at least 25 amino acids. For example, the linker may have a length of from 15 to 30 amino acids. In various embodiments, longer linkers of at least 15 amino acids can provide improvements in yield upon expression in Pichia pastoris.

In other embodiments, the linker is a physiologically-cleavable linker, such as a protease-cleavable linker. For example, the protease may be a coagulation pathway protease, such as activated Factor XII. In certain embodiments, the linker comprises the amino acid sequence of Factor XI (SEQ ID NO: 49) and/or prekallikrein (SEQ ID NO: 50 or 51) or a physiologically cleavable fragment thereof. In other embodiments, the linker includes a peptide sequence that is targeted for cleavage by a neutrophil specific protease, such as neutrophil elastase, cathepsin G, and proteinase 3.

In some embodiments, the DNASE variant (e.g., a variant of DNASE1 (D1), DNASE1-LIKE 1 (D1L1), DNASE1-LIKE 2 (D1L2), DNASE1-LIKE 3 Isoform 1 (D1L3), DNASE1-LIKE 3 Isoform 2 (D1L3-2), DNASE2A (D2A), and DNASE2B (D2B)) comprises an N-terminal or C-terminal fusion to a half-life extending moiety, such as albumin, transferrin, an Fc, or elastin-like protein. See U.S. Pat. No. 9,458,218, which is hereby incorporated by reference in its entirety. In some embodiments, the DNASE variant is dimerized by an immunoglobulin hinge region. For example, the engineered enzymes described herein may also include an Fc-fusion domain (e.g. a hinge and CH2 domains and CH3 domains of an immunoglobulin). In other cases, the engineered DNASE variant is fused to albumin, e.g., human albumin (SEQ ID NO: 12) or a fragment thereof. See WO 2015/066550; U.S. Pat. No. 9,221,896, which are hereby incorporated by reference in its entirety. Albumin can be fused at the N-terminus or the C-terminus of the engineered DNASE variant, and may optionally comprise an amino acid linker. In some embodiments, two DNASE variants are dimerized by an Fc hinge region, creating a dimeric molecule with synergistic functional properties for degrading NETs.

In some embodiments, human albumin and a flexible linker is fused to the N-terminus of DNASE1 (e.g., SEQ ID NO: 13), DNASE1-LIKE 1 (e.g., SEQ ID NO: 14), DNASE1-LIKE 2 (e.g., SEQ ID NO: 15), DNASE1-LIKE 3 Isoform 1 (e.g., SEQ ID NO: 16), DNASE1-LIKE 3 Isoform 2 (e.g., SEQ ID NO: 17), DNASE2A (e.g., SEQ ID NO: 18), and DNASE2B (e.g., SEQ ID NO: 19).

In some embodiments, the recombinant DNASE variant comprises one or more polyethylene glycol (PEG) moieties, which may be conjugated at one or more of positions or the C-terminus. In some embodiments, the native amino acid at that position is substituted with an amino acid having a side chain suitable for crosslinking with hydrophilic moieties, to facilitate linkage of the hydrophilic moiety to the peptide. In other embodiments, an amino acid modified to comprise a hydrophilic group is added to the peptide at the C-terminus. The PEG chain(s) may have a molecular weight in the range of about 500 to about 40,000 Daltons. In some embodiments, the PEG chain(s) have a molecular weight in the range of about 500 to about 5,000 Daltons. In some embodiments, the PEG chain(s) have a molecular weight of about 10,000 to about 20,000 Daltons.

The extracellular DNASE variants can be screened in assays for altered properties, including altered pH and temperature optimum, requirement for divalent cations for enzymatic activity, mechanisms of enzymatic inhibition (e.g. salt, divalent cations, actin, heparin, proteases), substrate affinity and specificity (e.g. single-stranded DNA, double-stranded DNA, chromatin, NETs, plasmid DNA, mitochondrial DNA), localization upon secretion (e.g. membrane-bound, extracellular matrix), localization signals (e.g. nuclear localization signal, membrane anchor), glycosylation sites, disulfide-bonds and unpaired cysteines, compatibility with GMP-compliant in vitro expression systems (e.g. bacteria. yeast, mammalian cells), compatibility with carriers (e.g. PEGylation, Fc fragment, albumin), compatibility with GMP-compliant purification methods (e.g. anion exchange resins, cation exchange resins), toxicological profile, tissue penetration, pharmacokinetics and pharmacodynamics.

In some embodiments, the DNASE variants are evaluated using an in vitro nucleic acid degradation assay, which can employ single or double-stranded DNA, plasmid DNA, mitochondrial DNA, NETs, or may employ chromatin. In some embodiments, the assay is a NET-degrading assay. The in vitro assay can be performed under different conditions including varying pH, temperature, divalent cations, and/or salt, to evaluate the enzyme characteristics for clinical applications. In some embodiments, enzyme activity is evaluated with fusion to carrier proteins such as albumin or Fc, or with PEGylation.

In some embodiments, the DNASE variants are evaluated for their expression potential in prokaryotic and/or eukaryotic (including mammalian and non-mammalian) expression systems, including their ease of expression, yield of recombinant enzyme, ability to be secreted as active protein, the lack of inclusion bodies, the presence of and identification of sites of glycosylation, and ease of purification with or without purification tags. In some embodiments, enzyme expression is evaluated with fusion to carrier proteins such as albumin or Fc. In some embodiments, DNASE variants are evaluated with substitution of any unpaired Cysteines.

In some embodiments, the DNASE variants are evaluated for short term and/or long term stability (e.g., upon storage for several months at 4° C. and/or room temperature). Stability can be evaluated by formation of aggregates, change of composition color, and/or enzyme activity.

In some embodiments, the DNASE variants are evaluated in animal models, including for immunogenic potential (e.g., presence of anti-DNASE variant antibodies), half-life in circulation, protease resistance, bioavailability, and/or NET-degrading activity. In some embodiments, activity is evaluated in disease models. Exemplary animal models may include rodent models (mouse, rat, rabbit) or primate models (e.g., chimpanzee).

In some embodiments, at least one DNASE variant is evaluated in a genetically modified mouse deficient in D1 and D1L3 activity, the mouse further having a heterologous expression of a G-CSF polynucleotide (e.g., in hepatocyte cells) or induction of a sustained endogenous G-CSF expression (e.g., via repetitive administration of microbial compounds). This mouse model accumulates NETs and rapidly develops NET-related vascular occlusions. In these embodiments, the invention comprises selecting DNASE enzyme that reduces the accumulation of NETs. The selected enzyme is formulated (as described) for administration to a human patient. One skilled in the art recognizes standard methods for generating double knockout Dnase1^{-/-}, Dnase1l3^{-/-} mice. Detailed descriptions can be found in, for example, U.S. Application Publication No. US 2019/0350178 and PCT International Patent Publication No. WO 2019/036719, the disclosure of which is incorporated herein by reference the in its entirety.

The invention further provides pharmaceutical compositions comprising extracellular DNASE variant as described herein, or optionally the polynucleotide or the vector as described, and a pharmaceutically acceptable carrier. The pharmaceutical composition may be formulated for any administration route, including topical, parenteral, or pulmonary administration. In various embodiments, the composition is formulated for intradermal, intramuscular, intraperitoneal, intraarticular, intravenous, subcutaneous, intraarterial, oral, sublingual, pulmonary, or transdermal administration.

In various embodiments, a selected DNASE variant is formulated with a “pharmaceutically acceptable carrier”, which includes any carrier that does not interfere with the effectiveness of the biological activity and is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

In other aspects, the invention provides a method for treating a subject in need of extracellular DNA degradation, extracellular chromatin degradation, extracellular trap (ET) degradation and/or neutrophil extracellular trap (NET) degradation. The method comprises administering a therapeutically effective amount of the extracellular DNASE variant or composition described herein. Exemplary indications where a subject is in need of extracellular DNA or chromatin degradation (including ET or NET degradation) are disclosed in PCT/US18/47084, the disclosure of which is appended to this application.

The invention is further described with reference to the following non-limiting examples.

### EXAMPLES

### Example 1: The Approach Used for Engineering DNASE Variants for Therapeutic Applications

DNASE1 (D1) forms along with DNASE1-LIKE 1 (D1L1), DNASE1-LIKE 2 (D1L2) and DNASE1-LIKE 3 (D1L3), the DNASE1-protein family, a group of homologous secreted DNase enzymes. DNASE2A and DNASE2B form an additional group of homologous DNase enzymes. DNASE1- and DNASE2-protein family members are evolutionary conserved and expressed in various species, including humans. In general, all extracellular DNASE enzymes provide drug candidates for therapies of diseases that are associated NETs. However, the physical, enzymatic, toxicological, and pharmacokinetic properties of these enzymes are not ideal for clinical applications.

An engineered D1 variant that is resistant to actin has been generated. Actin is an inhibitor of wild type D1. In brief, the 3D structure of the actin-DNASE1 complex was generated and actin binding sites in D1 were identified. Next, recombinant D1 variants with amino acids substitutions in the actin binding sites were expressed and tested for their sensitivity towards actin inhibition. The mutation A136F in SEQ ID NO: 1 was identified to generate the best actin-resistant D1 variants. See Ulmer et al., PNAS USA Vol. 93, pp 8225-8229 (1996).

Rats express a D1 variant that is naturally resistant to actin inhibition due to mutations in actin binding sites. Furthermore, the enzymatic activity of human D1L2 and D1L3 is not inhibited by actin. Indeed, human D1L3 features an F139, which corresponds to A136 in human D1 and likely causes the actin-resistance of D1L3.

Without being bound by theory, it was proposed that enzymatic properties that are favorable for development of therapy with extracellular DNASE enzymes can be transferred to human extracellular DNASE enzymes from extracellular DNASE enzymes expressed in other species (e.g. rat) or from other members of the same extracellular DNASE protein family (e.g. DNASE1-protein family comprised of DNASE1 (D1), DNASE1-LIKE 1 (D1L1), DNASE1-LIKE 2 (D1L2), DNASE1-LIKE 3 Isoform 1 (D1L3), DNASE1-LIKE 3 Isoform 2 (D1L3-2), and DNASE1-protein family comprised of DNASE2A (D2A), and DNASE2B (D2B)).

A protein engineering technology, termed Building Block Protein Engineering, can be applied to members of the DNASE1 and DNASE2 protein family and an extracellular DNASE (e.g. D1, D1L1, D1L2, D1L3, D1L3-2, D2A, D2B). Building Block Protein Engineering is based on the following steps: providing a protein-protein alignment of donor and recipient DNASE enzymes; identifying variable amino acid(s) for transfer, the variable amino acid(s) being flanked by one or more conserved amino acids in the donor and recipient DNase enzymes; substituting the variable amino acid(s) of the recipient DNase with the variable amino acid(s) of the donor DNase to create a chimeric DNase; and recombinantly producing the chimeric DNase.

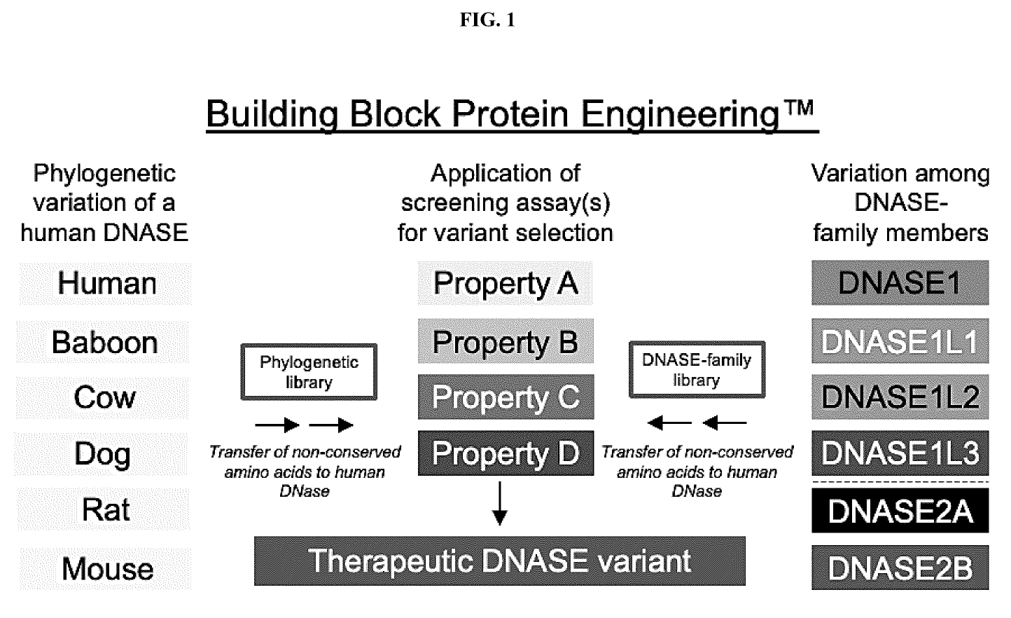

This approach can generate two distinct types of libraries with variants of extracellular DNASE enzymes: a library based on phylogenetic variation of a human extracellular DNASE, and a library that is based on variation among DNASE-family members (FIG. 1).

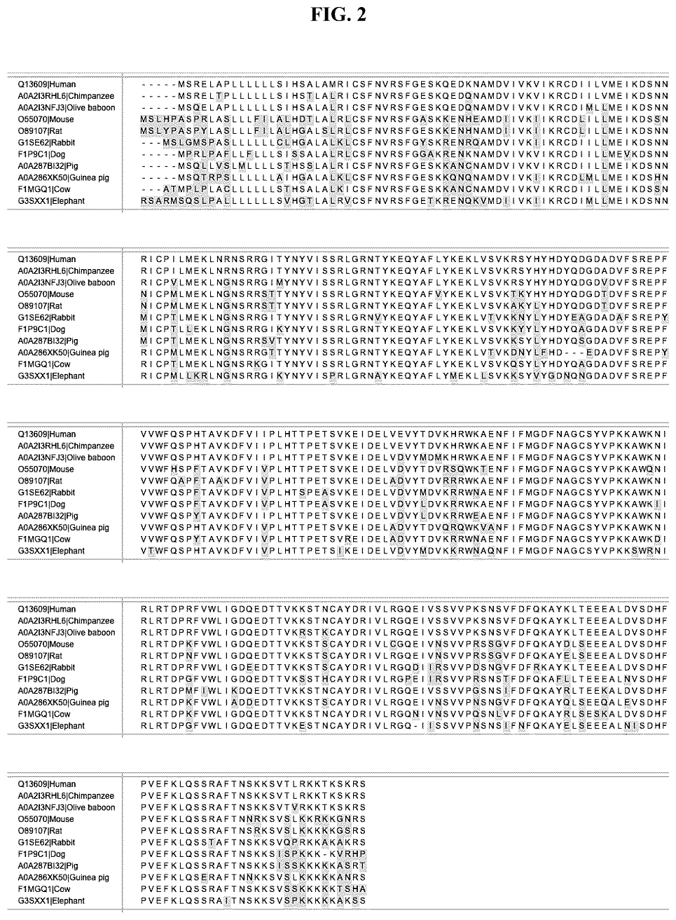

For example, FIG. 2 shows the alignment of human D1L3, with D1L3 from other species, including chimpanzee, baboon, mouse, rat, rabbit, dog, pig, guinea pig, cow, and elephant, and which identifies non-conserved amino acids (Building Blocks) that when transferred to human D1L3 result in phylogenetic variants of human D1L3. These feature the following mutations: M21L, K22R, I22L, I22V, E33A, E33Y, E33G, S34A, S34T, Q36K, Q36R, E37A, E37Q, D48N, K39Q, K39H, K39R, K39C, N40E, N40Q, N40K, A41V, V44I, V53I, I53L, I54M, V57L, I60V, N64S, H64S, R66N, R66M, I70V, I70M, I70T, M72L, E73K, K74R, R77G, R81K, G82S, I83V, I83T, T84M, T84K, S91P, T91V, T91A, L105V, K107M, V111L, S112T, R115T, R115A, R115K, R115D, R115Q, S116K, S116N, S116Y, H118L, H118V, Y119F, H120G, Y122N, Q123E, D124A, D124S, D124N, G125E, A127V, A127T, V129A, F135Y, V137T, Q140H, S141A, H143F, H143Y, V146A, I152V, T157S, T160A, V162I, K163R, V169A, E170D, T173M, T173L, V175M, K176R, K176Q, H177S, H177R, R178Q, K180E, K180N, K181T, K181V, E183A, E183Q, A201S, K203Q, K203R, R212K, R212N, R212G, R212M, V214I, G218K, G218A, Q220E, Q220D, K227R, K227S, K227E, N239K, N239S, N239H, R239C, Q241P, E242D, E242N, V244I, S245N, S245R, K250R, K250D, K250R, K250G, K250N, K250Q, N252S, S253G, S253L, V254T, V254I, D256N, Q258R, Y261F, K262D, K262E, K262L, K262R, K262Q, T264S, E266S, E267K, E267Q, E267K, D270N, D270E, V271I, S282E, R285T, F287I, S290N, K291R, V294I, T295S, T295Q, L296V, L296P, L296S, R297K, K299R, T300K, T300A, S302G, S302A, S302V, S302T, K303N, K303S, K303R, R304H, R304S, S305P, S305T, and S305A.

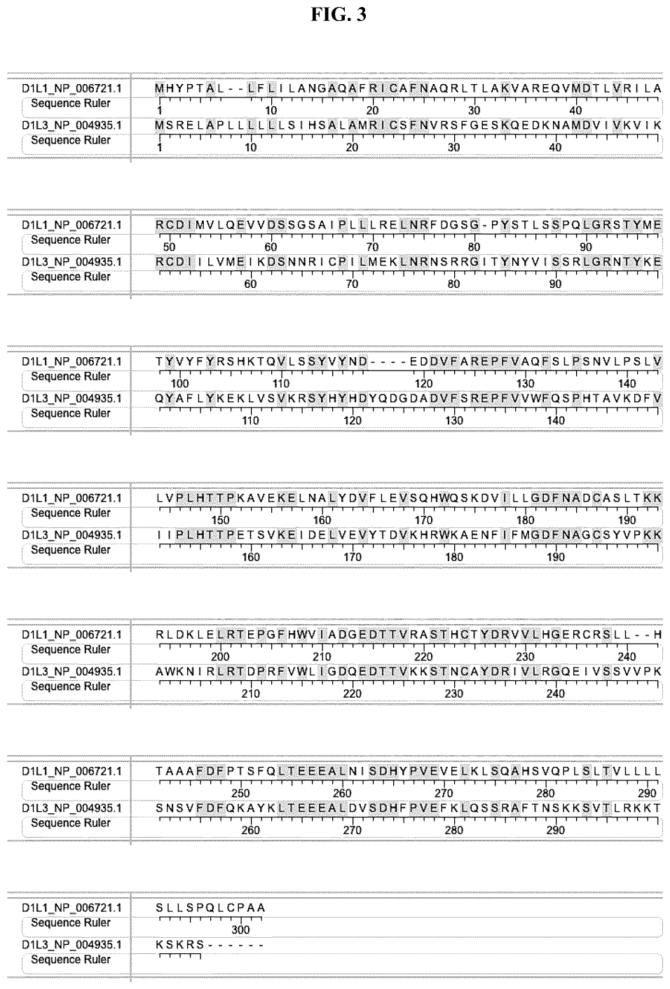

FIG. 3 shows the alignment of human D1L3 with human D1L1, and identifies non-conserved amino acids (Building Blocks) that when transferred to human D1L3 result in variants of human D1L3. These feature the following mutations: S2_L5delinsHYPT, P7_L9delinsL, L11F, L13_S17delinsILANG, L19delinsQ, M21F, S25A, V28_S34delinsAQRLTLA, Q36_A41delinsVAREQV, V44_I45delinsTL, K47_K50delinsRILA, I55_M58delinsMVLQ, I60_K61delinsVV, N6_C68delinsSGSAI, I70L, M72_L74delinsLRE, N78_R81delinsFDGS, I83_T84delinsP, N86_I89delinsSTLS, S91_R92delinsPQ, N96S, K99M, Q101T, A103_L105delinsVYF, K107_S112delinsRSHKTQ, K114_R115delinsLS, H118V, H120N, Y122_A127delinsED, S131A, V137_W138delinsAQ, Q140_S141delinsSL, H143_F149delinsSNVLPSL, I151_I152delinsLV, E159_V162delinsKAVE, I165_A167delinsLNA, V169_E170delinsYD, Y172_D174delinsFLE, K176_R178delinsSQH, K180_F184delinsQSKDV, G193D, S195_P198delinsASLT, A201_R206delinsRLDKLE, D210E, R212G, V214H, L216V, G218A, Q220G, K226_K227delinsRA, N230H, A232T, I236V, R239H, Q246_V249delinsERCR, S246_V254delinsLLHTAAA, Q258_K262delinsPTSFQ, D270_V271delinsNI, F275Y, F279_K280delinsVE, Q282_S283delinsKL, R285Q, F287_K292delinsHSVQPL, V294L, L296_S205delinsVLLLLSLLSPQLCPAA.

Such extracellular DNASE variants can be screened in assays for altered properties, including altered pH and temperature optimum, requirement for divalent cations for enzymatic activity, mechanisms of enzymatic inhibition (e.g. salt, divalent cations, actin, heparin, proteases), substrate affinity and specificity (e.g. single-stranded DNA, double-stranded DNA, chromatin, NETs, plasmid DNA, mitochondrial DNA), localization upon secretion (e.g. membrane-bound, extracellular matrix), localization signals (e.g. nuclear localization signal, membrane anchor), glycosylation sites, disulfide-bonds and unpaired cysteines, compatibility with GMP-compliant in vitro expression systems (e.g. bacteria. yeast, mammalian cells), compatibility with carriers (e.g. PEGylation, Fc fragment, albumin), compatibility with GMP-compliant purification methods (e.g. anion exchange resins, cation exchange resins), toxicological profile, tissue penetration, pharmacokinetics and pharmacodynamics.

An extracellular DNASE with altered profile can provide a drug candidate for diseases that are associated with NETs.

### Example 2: Development of Building Block Engineering of DNase1-Protein Family Members

D1L3 features three sites that contain additional amino acids: the C-terminal tail starting after Q282 (NH2-SSRAFTBSKKSVTLRKKTKSKRS-COOH) (SEQ ID NO: 21), and at two sites within the enzyme at S79/R80 and at K226. The 23 amino acids of the C-terminal tail of D1L3 have been attached to the C-terminus of D1. It was observed that the insertion of an arginine-residue at position 226 of DNase1 (A226_T227insK) generated a D1-variant with reduced enzymatic activity to degrade dsDNA, while no such effect was observed with the substitution T227K. Thus, an insertion of a K/R-residue goes along with a risk of reducing D1 function. The insertion of a charged amino acid may influence the local protein structure. Given that D1 is a globular enzyme that comprises one amino acid chain, it is conceivable that such local alteration may render the whole enzyme inactive. Indeed, numerous non-conservative mutations throughout the D1 amino acid sequence inactivate the enzyme. Without being bound by theory, it was hypothesized that the transfer of local protein structures by implanting not only single arginine and lysine residues but also the neighboring amino acids sequences reduces the risk of inactivation. Conserved amino acids were searched within D1 and D1L3 for in the vicinity of A226_T227insK that can be used as anchors for the insertion. A D223/T224/T225 motif and a conserved T229 in D1 as N-terminal and C-terminal anchors, respectively were identified. 3 amino acids within D1 (ATP) were replaced with 4 amino acids, including K226, from D1L3 (VKKS) in silico. Expression of the cDNA of the new D1-variant (A226_P228delinsVKKS) in HEK239 cells revealed a functionally active enzyme with a similar dsDNA-degrading activity, when compared to wild-type D1. The data suggest that the variable amino acids between conserved amino acids are interchangeable between D1 and D1L3.

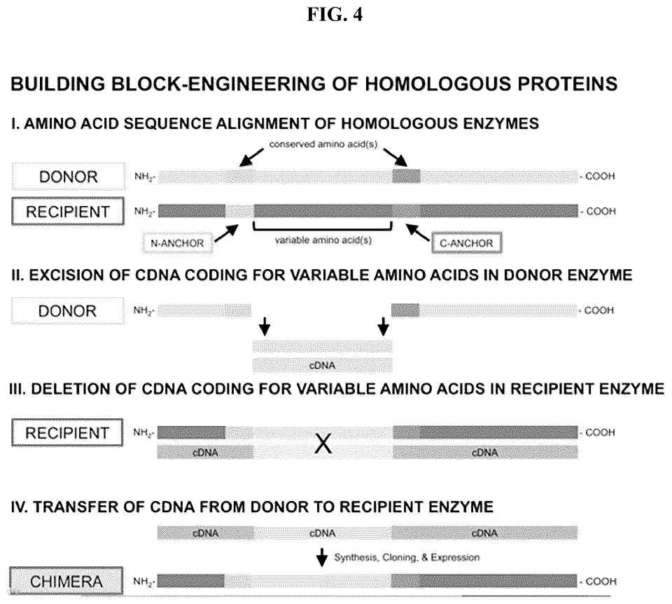

We conceptualized a building block-technology to transfer enzymatic properties from one member of the D1-protein family to another. The following cardinal steps characterize the technology (FIG. 4):
(1) Provide protein-protein alignment of donor and recipient DNase
(2) Identify variable amino acid or amino acid sequence for transfer (building block)
(3) Identify conserved amino acids in donor and recipient DNase that are located up and downstream of building block (anchors), respectively.
(4) Replace cDNA encoding for building block between C- and N-anchors in recipient DNase, with cDNA between the anchors in donor DNase.
(5) Synthesize cDNA of chimeric DNase, followed by in vitro/in vivo expression into a recipient organism that is preferably deficient in both donor and recipient DNase (e.g. CHO cells or Dnase1^{-/-}Dnase1l3^{-/-} mice).

### Example 3: Engineering DNase1 Variants Through Building Block Technology

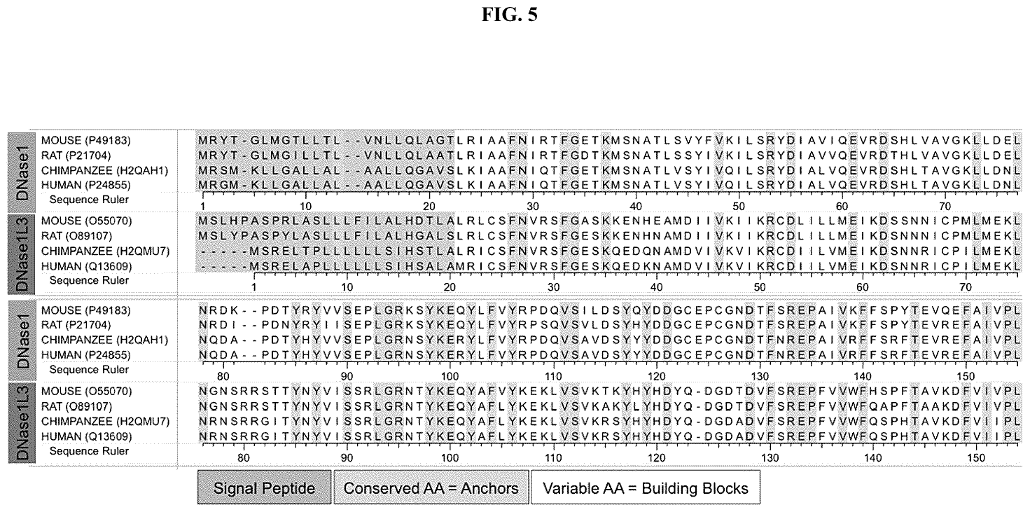

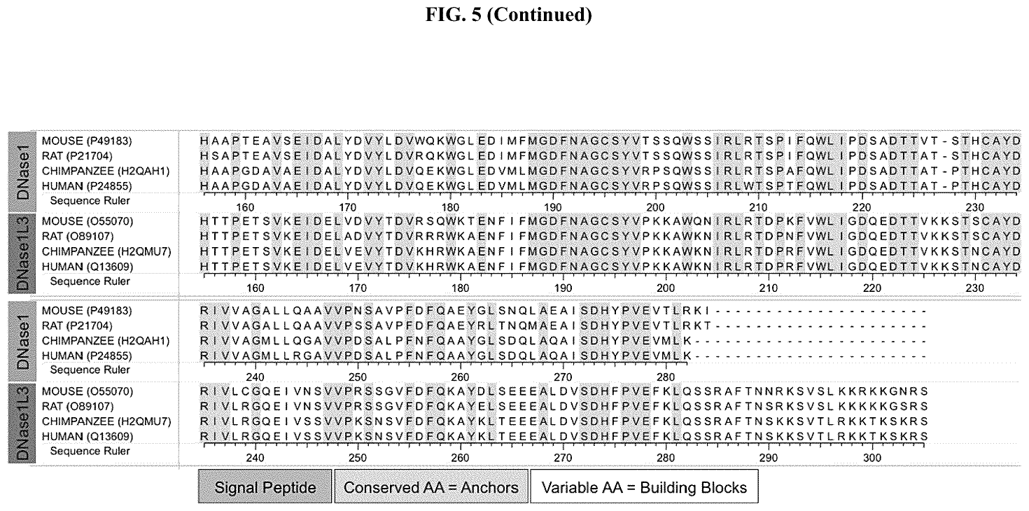

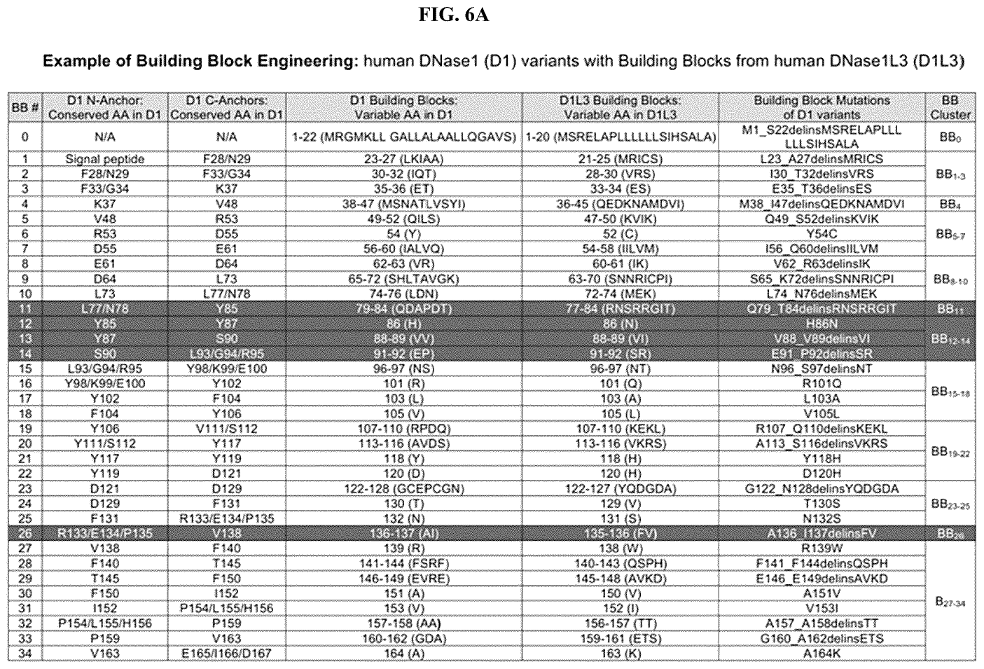

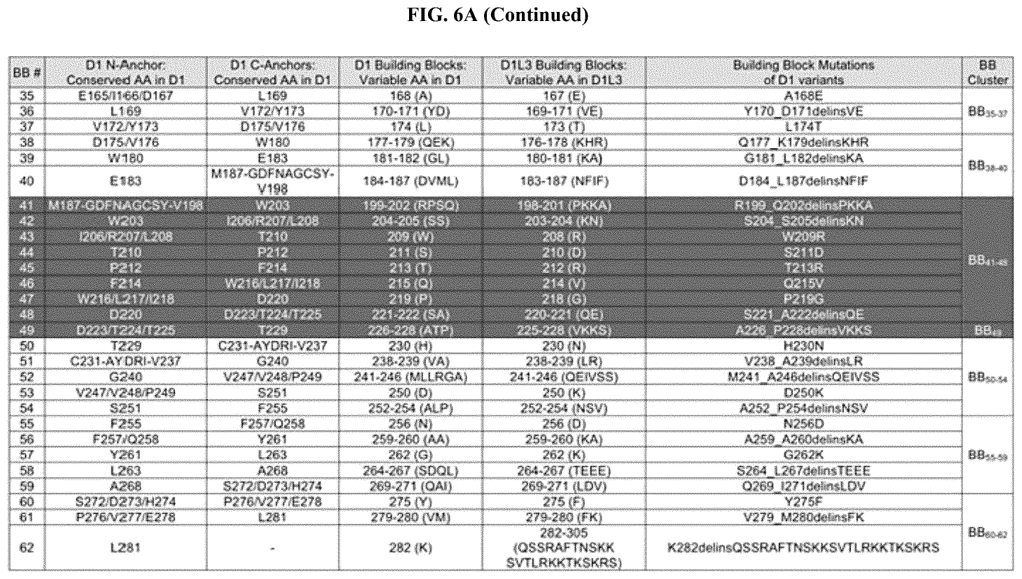

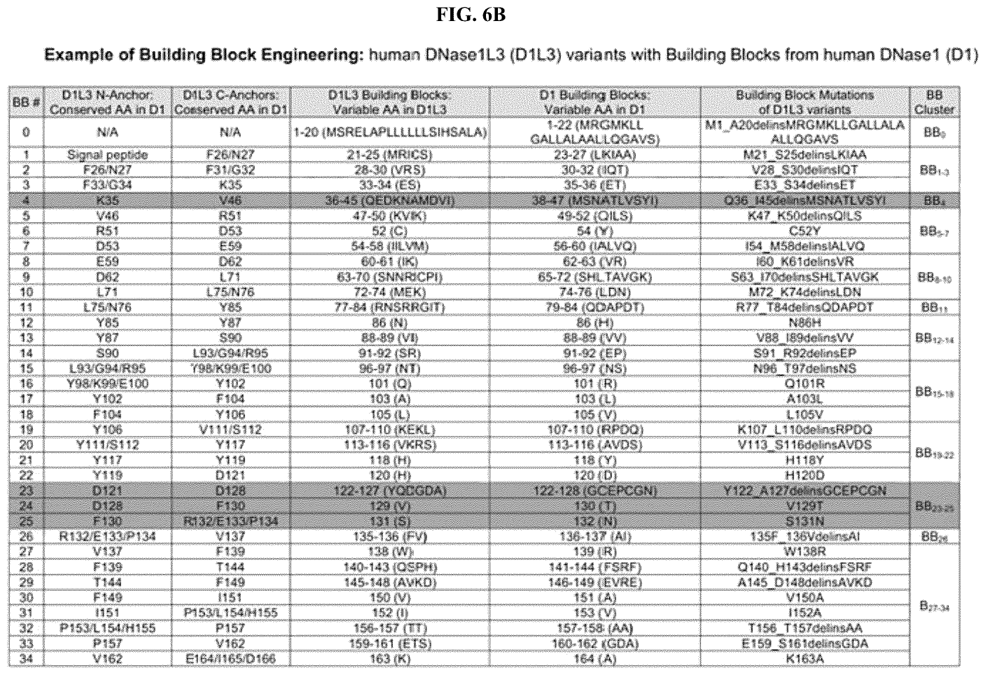

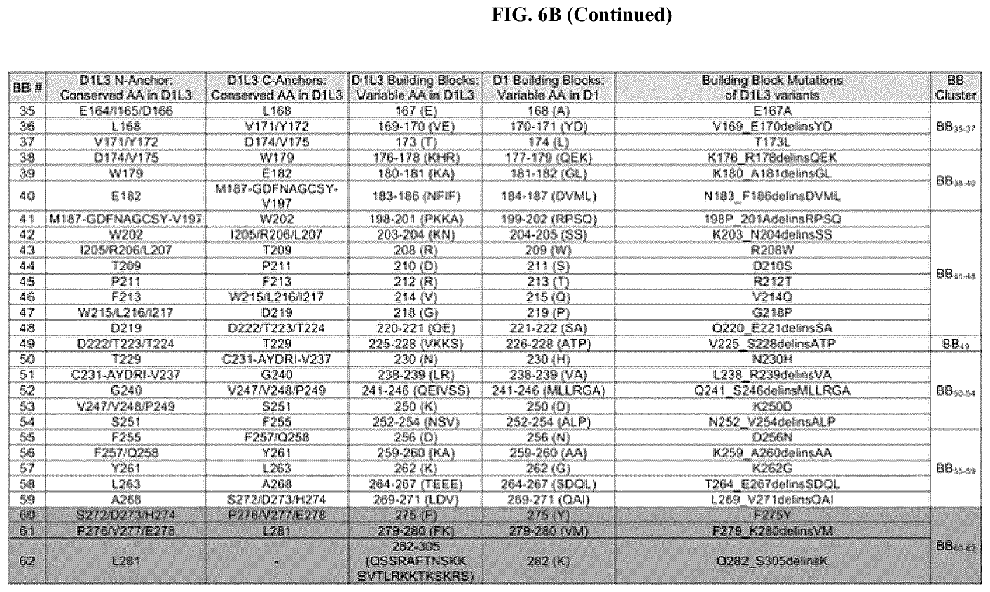

A multiple-species alignment of D1 and D1L3 from human, mouse, rat, and chimpanzee (FIG. 5), showed that N- and C-terminal anchors are conserved among these species. These anchor amino acids or amino acid sequences flank 62 building blocks of variable amino acids and amino acid sequences, which include the amino acid sequence in D1 (ATP) and D1L3 (VKKS) from building blocks #49 (FIGS. 6A-6B).

The transfer of these building blocks from D1L3 into D1 generates D1-variants with the following mutations (FIGS. 6A-6B): 1M_S22delinsMSRELAPLLLLLLSIHSALA, L23_A27delinsMRICS, I30_T32delinsVRS, E35_T36delinsES, M38_I47delinsQEDKNAMDVI, Q49_S52delinsKVIK, Y54C, I56_Q60delinsIILVM, V62_R63delinsIK, S65_K72delinsSNNRICPI, L74_N76delinsMEK, Q79_T84delinsRNSRRGIT, H86N, V88_V89delinsVI, E91_P92delinsSR, N96_S97delinsNT, R101Q, L103A, V105L, R107_Q110delinsKEKL, A113_S116delinsVKRS, Y118H, D120H, G122_N128delinsYQDGDA, T130S, N132S, A136_I137delinsFV, R139W, F141_F144delinsQSPH, E146_E149delinsAVKD, A151V, V153I, A157_A158delinsTT, G160_A162delinsETS, A164K, A168E, Y170_D171delinsVE, L174T, Q177_K179delinsKHR, G181_L 182delinsKA, D184_L187delinsNFIF, R199_Q202delinsPKKA, S204_S205delinsKN, W209R, S211D, T213R, Q215V, P219G, S221_A222delinsQE, A226_P228delinsVKKS, H230N, V238_A239delinsLR, M241_A246delinsQEIVSS, D250K, A252_P254delinsNSV, N256D, A259_A260delinsKA, G262K, S264_L267delinsTEEE, Q269_I271delinsLDV, Y275F, V279_M280delinsFK, and K282delinsQSSRAFTNSKKSVTLRKKTKSKRS.

The following D1L3-variants are generated if the building blocks are transferred from D1 to D1L3 (FIGS. 6A-6B): M1_A20delinsMRGMKLLGALLALAALLQGAVS, M21_S25delinsLKIAA, V28_S30delinsIQT, E33_S34delinsET, Q36_I45delinsMSNATLVSYI, K47_K50delinsQILS, C52Y, I54_M58delinsIALVQ, I60_K61delinsVR, S63_I70delinsSHLTAVGK, M72_K74delinsLDN, R77_T84delinsQDAPDT, N86H, V88_I89delinsVV, S91_R92delinsEP, N96_T97delinsNS, Q101R, A103L, L105V, K107_L110delinsRPDQ, V113_S116delinsAVDS, H118Y, H120D, Y122_A127delinsGCEPCGN, V129T, S131N, 135F_136VdelinsAI, W138R, Q140_H143delinsFSRF, A145_D148delinsAVKD, V150A, I152A, T156_T157delinsAA, E159_S161delinsGDA, K163A, E167A, V169_E170delinsYD, T173L, K176_R178delinsQEK, K180_A181delinsGL, N183_F186delinsDVML, P198_A201delinsRPSQ, K203_N204delinsSS, R208W, D210S, R212T, V214Q, G218P, Q220_E221delinsSA, V225_S228delinsATP, N230H, L238_R239delinsVA, Q241_S246delinsMLLRGA, K250D, N252_V254delinsALP, D256N, K259_A260delinsAA, K262G, T264_E267delinsSDQL, L269_V271delinsQAI, F275Y, F279_K280delinsVM, and Q282_S305delinsK.

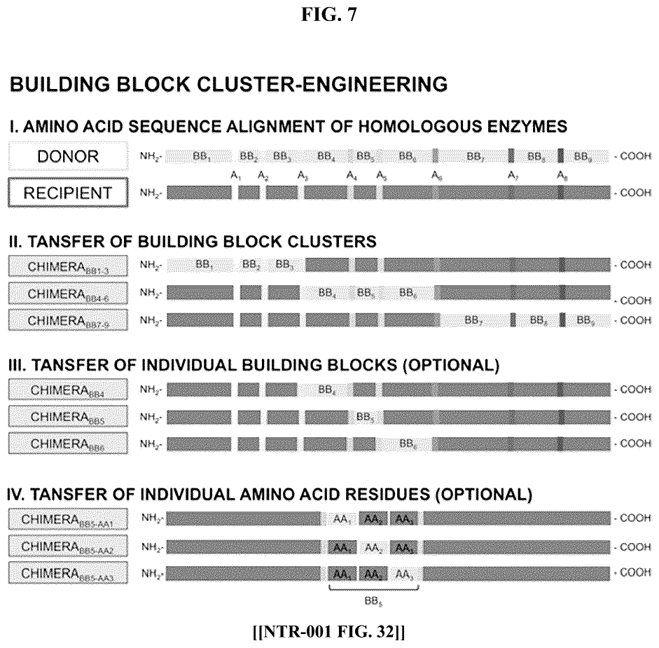

Next, we conceptualized a sequential approach to engineer D1-variants with D1L3 activity that starts with the transfer of multiple adjacent building blocks (clusters), continues with the transfer of individual building blocks, and ends with a transfer of individual amino acids or the combination of multiple building blocks into new chimeric enzymes (FIG. 7). This approach reduces the number of D1-D1L3-chimera in the initial screening.

To test our method, we designed a total of 19 D1-variants comprising either individual building blocks or clusters of building block cluster from D1L3 (FIG. 6). These D1-variants feature the following amino acid mutations: 1M_S22delinsMSRELAPLLLLLLSIHSALA, L23_A27delinsMRICS/I30_T32delinsVRS/E35_T36delinsES, M38_I47delinsQEDKNAMDVI, Q49_S52delinsKVIK/Y54C/I56_Q60delinsIILVM, V62 R63delinsIK/S65_K72delinsSNNRICPI/L74_N76delinsMEK, Q79_T84delinsRNSRRGIT, H86N/V88_V89delinsVI/E91_P92delinsSR, N96_S97delinsNT/R101Q/L103A/V105L, R107_Q110delinsKEKIUA 113_S116delinsVKRS/Y118H/D120H, G122_N128delinsYQDGDA/T30S/N132S, A136_I137delinsFV, R139W/F141_F144delinsQSPH/E146_E149delinsAVKD/A151VN153I/A157_A158d elinsTT/G160_A162delinsETS/A164K, A168E/Y170_D171delinsVE/L174T, Q177_K179delinsKHR/G181_L 182delinsKA/D184_L187delinsNFIF, R199_Q202delinsPKKA/S204_S205delinsKN/W209R/S21ID/T213R/Q215V/P219G/S221_A222delinsQE, A226_P228delinsVKKS, H230N/V238_A239delinsLR/M241_A246delinsQEIVSS/D250K/A252_P254delinsNS V, N256D/A259_A260delinsKA/G262K/S264_L267delinsTEEE/Q269_I271delinsLDV, and Y275FN279_M280delinsFK/K282delinsQSSRAFTNSKKSVTLRKKTKSKRS.

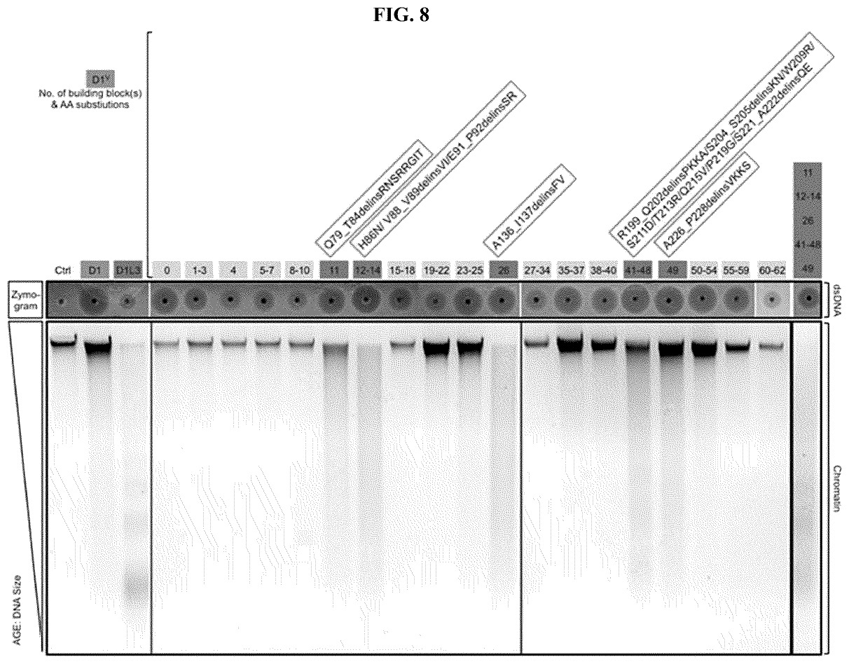

Next, we cloned the cDNA into an expression vector, which was transfected into HEK293 cells. Analysis of the cell supernatants showed dsDNA degradation by all samples (FIG. 8). Furthermore, we observed that the transfer of building blocks (BB) 11, BB 12-14, BB 26, BB 41-48, and BB 49 from D1L3 to D1 resulted in enzymes with increased chromatin degrading activity. All these chimeric enzymes exhibited the same or more activity to degrade dsDNA substrates than wild-type D1. The building blocks 11 and 49 from D1L3 contain R80/R81 and K227, respectively, which are not present in D1. The D1L3-BB cluster 41-48 features 5 additional arginine and lysine residues than its counterpart in D1. These additional cationic amino acids may be responsible for the hyperactivity. The D1-building blocks 12-14 and 26 contain the amino acid sequences H86 to R95 and A136 to V138 in SEQ ID NO: 1, which includes amino acid residues that are required for binding of the D1-inhibitor actin. Thus, replacement of these amino acid sequences with the respective building blocks from D1L3, which do not interact with actin, likely generates actin-resistant variants of D1. We now combined BB 11, 14, 26, 41-19 in one novel D1-variant. We observed that the combination of these gain-of-function BBs increased the chromatin degrading of the D1 variant to levels of wild-type D1L3 (FIG. 8). Thus, the BB technology provides a robust method to generate hyperactive D1 variants.

### Example 4: Expression and Characterization of D1L3 with Basic Domain Deletion (BDD) in Chinese Hamster Ovarian (CHO) Cells and in Pichia pastoris

DNASE1 and DNASE1L3 preferentially cleave protein-free DNA and DNA-histone-complexes (i.e. chromatin), respectively. Previous studies suggest that a basic domain (BD) at the C-terminus of DNASE1L3, which is absent in DNASE1, is responsible for the distinct substrate specificities of both enzymes (Sisirak et al., Cell, 2016; Keyel, Developmental Biology, 2017).

To characterize the amino acids that are responsible for chromatin-degrading activity (“chromatinase” activity), wild-type D1L3 was substituted with building block substitutions from D1, as disclosed in PCT/US2018/047084. The building block substitutions to D1L3 are selected from human D1 and result in variants of human D1L3, which feature the following mutations: M21_R22delinsLK, C24_S25delinsAA, V28_S30delinsIQT, S34T, Q36_V44delinsMSNATLVSY, K47_K50delinsQILS, C52Y, I55_M58delinsIALVQE, I60_K61delinsVR, N64_I70delinsHLTAVGK, M72_K74delinsLDN, R77_I83delinsQDAPD, N86H, I89V, S91_R92delinsEP, T97S, Q101R, A103L, L105V, K107_L110delinsRPDQ, V113_R115delinsAVD, H118Y, H120D, Y122_A127delinsGCEPCGN, V129T, S131N, F135_V136delinsAI, W138R, Q140_H143delinFSRF, A145_D148delinsEVRE, V150A, I152V, T156_T157delinsAA, E159_S161delinsGDA, K163A, E167A, V169_E170delinsYD, T173L, K176_R178delinsQEK, K180_A181delinsGL, N183_F186delinsDVML, P198_A201delinsRPSQ, K203_N204delinsSS, R208W, D210S, R212T, V214Q, G218P, Q220_E221delinsSA, V225_S228delinsATP, N230H, L238_R239delinsVA, Q241_S246delinsMLLRGA, K250D, N252_V254delinsALP, D256N, K259A, K262G, T264_E267delinsSDQL, L269_V271delinsQAI, F275Y, F279_K280delinsVM, Q282_S205delinsK with respect to human D1L3, Isoform 1.

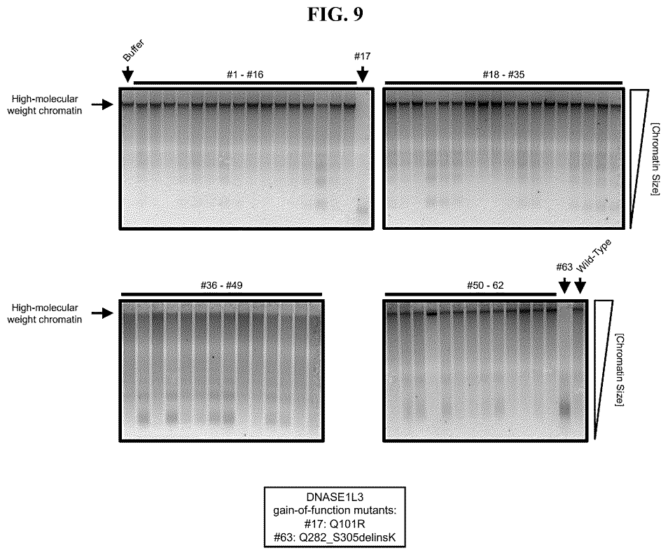

These 63 D1L3variants were screened for loss or gain of chromatin-degrading activity. In brief, D1L3 variants were transiently expressed in CHO cells using an in vitro expression vector. Culture supernatants were collected and tested for chromatin-degrading activity using purified nuclei as a source of chromatin. As shown in FIG. 9, the building block substitution #63 from D1 significantly improved the degradation of high-molecular weight (HMW) chromatin to small fragments, when compared to wild-type D1L3. Building block substitution #63 causes the mutation Q282_S305delinsK, which deletes the full C-terminal BD of D1L3 from amino acid position 283 to 305 and replaces glutamine (Q) at position 282 with lysine.

### INCORPORATION BY REFERENCE

All patents and publications referenced herein are hereby incorporated by reference in their entireties.

DNASE1 (NP_005212.2): Signal Peptide, Mature
Protein:
SEQ ID NO: 1
MRGMKLLGALLALAALLQGAVSLKIAAFNIQTFGETKMSNATLVSYIVQI
LSRYDIALVQEVRDSHLTAVGKLLDNLNQDAPDTYHYVVSEPLGRNSYKE
RYLFVYRPDQVSAVDSYYYDDGCEPCGNDTFNREPAIVRFFSRFTEVREF
AIVPLHAAPGDAVAEIDALYDVYLDVQEKWGLEDVMLMGDFNAGCSYVRP
SQWSSIRLWTSPTFQWLIPDSADTTATPTHCAYDRIVVAGMLLRGAVVPD
SALPFNFQAAYGLSDQLAQAISDHYPVEVMLK
DNASE1-LIKE 1 (NP_006721.1): Signal Peptide;
Mature Protein:
SEQ ID NO: 2
MHYPTALLFLILANGAQAFRICAFNAQRLTLAKVAREQVMDTLVRILARC
DIMVLQEVVDSSGSAIPLLLRELNRFDGSGPYSTLSSPQLGRSTYMETYV
YFYRSHKTQVLSSYVYNDEDDVFAREPFVAQFSLPSNVLPSLVLVPLHTT
PKAVEKELNALYDVFLEVSQHWQSKDVILLGDFNADCASLTKKRLDKLEL
RTEPGFHWVIADGEDTTVRASTHCTYDRVVLHGERCRSLLHTAAAFDFPT
SFQLTEEEALNISDHYPVEVELKLSQAHSVQPLSLTVLLLLSLLSPQLCP
AA
DNASE1-LIKE 2 (NP_001365.1): Signal Peptide,
Mature Protein:
SEQ ID NO: 3
MGGPRALLAALWALEAAGTAALRIGAFNIQSFGDSKVSDPACGSILAKIL
AGYDLALVQEVRDPDLSAVSALMEQINSVSEHEYSFVSSQPLGRDQYKEM
YLFVYRKDAVSVVDTYLYPDPEDVFSREPFVVKFSAPGTGERAPPLPSRR
ALTPPPLPAAAQNLVLIPLHAAPHQAVAEIDALYDVYLDVIDKWGTDDML
FLGDFNADCSYVRAQDWAAIRLRSSEVFKWLIPDSADTTVGNSDCAYDRI
VACGARLRRSLKPQSATVHDFQEEFGLDQTQALAISDHFPVEVTLKFHR
DNASE1-LIKE 3; Isoform 1 (NP_004935.1): Signal
Peptide, Mature Protein:
SEQ ID NO: 4
MSRELAPLLLLLLSIHSALAMRICSFNVRSFGESKQEDKNAMDVIVKVIK
RCDIILVMEIKDSNNRICPILMEKLNRNSRRGITYNYVISSRLGRNTYKE
QYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKK
AWKNIRLRTDPRFVWLIGDQEDTTVKKSTNCAYDRIVLRGQEIVSSVVPK
SNSVFDFQKAYKLTEEEALDVSDHFPVEFKLQSSRAFTNSKKSVTLRKKT
KSKRS
DNASE1-LIKE 3, Isoform 2 (NP_001243489.1): Signal
Peptide; Mature Protein:
SEQ ID NO: 5
MSRELAPLLLLLLSIHSALAMRICSFNVRSFGESKQEDKNAMDVIVKVIK
RCDIILVMEIKDSNNRICPILMEKLNREKLVSVKRSYHYHDYQDGDADVF
SREPFVVWFQSPHTAVKDFVIIPLHTTPETSVKEIDELVEVYTDVKHRWK
AENFIFMGDFNAGCSYVPKKAWKNIRLRTDPRFVWLIGDQEDTTVKKSTN
CAYDRIVLRGQEIVSSVVPKSNSVFDFQKAYKLTEEEALDVSDHFPVEFK
LQSSRAFTNSKKSVTLRKKTKSKRS
DNASE2A (O00115): Signal Peptide; Mature Protein:
SEQ ID NO: 6
MIPLLLAALLCVPAGALTCYGDSGQPVDWFVVYKLPALRGSGEAAQRGLQ
YKYLDESSGGWRDGRALINSPEGAVGRSLQPLYRSNTSQLAFLLYNDQPP
QPSKAQDSSMRGHTKGVLLLDHDGGFWLVHSVPNFPPPASSAAYSWPHSA
CTYGQTLLCVSFPFAQFSKMGKQLTYTYPWVYNYQLEGIFAQEFPDLENV
VKGHHVSQEPWNSSITLTSQAGAVFQSFAKFSKFGDDLYSGWLAAALGTN
LQVQFWHKTVGILPSNCSDIWQVLNVNQIAFPGPAGPSFNSTEDHSKWCV
SPKGPWICVGDMNRNQGEEQRGGGILCAQLPALWKAFQPLVKNYQPCNGM
ARKPSRAYKI
DNASE2B (Q8WZ79): Signal Peptide; Mature Protein:
SEQ ID NO: 7
MKQKMMARLLRTSFALLFLGLFGVLGAATISCRNEEGKAVDWFTFYKLPK
RQNKESGETGLEYLYLDSTTRSWRKSEQLMNDTKSVLGRTLQQLYEAYAS
KSNNTAYLIYNDGVPKPVNYSRKYGHTKGLLLWNRVQGFWLIHSIPQFPP
IPEEGYDYPPTGRRNGQSGICITFKYNQYEAIDSQLLVCNPNVYSCSIPA
TFHQELIHMPQLCTRASSSEIPGRLLTTLQSAQGQKFLHFAKSDSFLDDI
FAAWMAQRLKTHLLTETWQRKRQELPSNCSLPYHVYNIKAIKLSRHSYFS
SYQDHAKWCISQKGTKNRWTCIGDLNRSPHQAFRSGGFICTQNWQIYQAF
QGLVLYYESCK

Selected Amino Acid Sequences of Human Wild-Type
DNASES
C-terminal tail of human DNASE1-LIKE 1
(NP_006721.1):
SEQ ID NO: 8
KLSQAHSVQPLSLTVLLLLSLLSPQLCPAA
Proline-rich extension of human DNASE1-LIKE 2
(NP_001365.1):
SEQ ID NO: 9
SAPGTGERAPPLPSRRALTPPPLPAAAQNLVLIPL
C-terminal tail of human DNASE1-LIKE 3; Isoform 1
(NP_004935.1):
SEQ ID NO: 10
SSRAFTNSKKSVTLRKKTKSKRS
Internal sequence of human DNASE1-LIKE 3; Absent
in Isoform 2 (NP_004935.1):
SEQ ID NO: 11
RNSRRGITYNYVISSRLGRNTYKEQYAFLYK

Carrier Protein
Human Albumin (P02768): Signal Peptide +
Propeptide; Mature Protein:
SEQ ID NO: 12
MKWVTFISLLFLFSSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDE
FKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL

Amino Acid Sequences of Human ALBUMIN-DNASE-Fusion proteins
Albumin-Linker-DNASE1;
Signal Peptide + Propeptide, Albumin, Flexible Linker, mature
DNASE1:
SEQ ID NO: 13
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSLKIAAFNIQTFGET
KMSNATLVSYIVQILSRYDIALVQEVRDSHLTAVGKLLDNLNQDAPDTYHYVVSEPLG
RNSYKERYLFVYRPDQVSAVDSYYYDDGCEPCGNDTFNREPAIVRFFSRFTEVREFAI
VPLHAAPGDAVAEIDALYDVYLDVQEKWGLEDVMLMGDFNAGCSYVRPSQWSSIRLWT
SPTFQWLIPDSADTTATPTHCAYDRIVVAGMLLRGAVVPDSALPFNFQAAYGLSDQLA
QATSDHYPVEVMLK
Albumin-Linker-DNASE1-LIKE 1;
Signal Peptide + Propeptide, Albumin, Flexible Linker, mature
DNASE1-LIKE 1:
SEQ ID NO: 14
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSFRICAFNAQRLTLA
KVAREQVMDTLVRILARCDIMVLQEVVDSSGSAIPLLLRELNRFDGSGPYSTLSSPQL
GRSTYMETYVYFYRSHKTQVLSSYVYNDEDDVFAREPFVAQFSLPSNVLPSLVLVPLH
TTPKAVEKELNALYDVFLEVSQHWQSKDVILLGDFNADCASLTKKRLDKLELRTEPGF
HWVIADGEDTTVRASTHCTYDRVVLHGERCRSLLHTAAAFDFPTSFQLTEEEALNISD
HYPVEVELKLSQAHSVQPLSLTVLLLLSLLSPQLCPAA
Albumin-Linker-DNASE1-LIKE 2;
Signal Peptide + Propeptide, Albumin, Flexible Linker, mature
DNASE1-LIKE 2:
SEQ ID NO: 15
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSLRIGAFNIQSFGDS
KVSDPACGSIIAKILAGYDLALVQEVRDPDLSAVSALMEQINSVSEHEYSFVSSQPLG
RDQYKEMYLFVYRKDAVSVVDTYLYPDPEDVFSREPFVVKFSAPGTGERAPPLPSRRA
LTPPPLPAAAQNLVLIPLHAAPHQAVAEIDALYDVYLDVIDKWGTDDMLFLGDFNADC
SYVRAQDWAAIRLRSSEVFKWLIPDSADTTVGNSDCAYDRIVACGARLRRSLKPQSAT
VHDFQEEFGLDQTQALAISDHFPVEVTLKFHR
Albumin-Linker-DNASE1-LIKE 3;
Signal Peptide + Propeptide, Albumin, Flexible Linker, mature
DNASE1-LIKE 3:
SEQ ID NO: 16
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSMRICSFNVRSFGES
KQEDKNAMDVIVKVIKRCDIILVMEIKDSNNRICPILMEKLNRNSRRGITYNYVISSR
LGRNTYKEQYAFLYKEKLVSVKRSYHYHDYQDGDADVFSREPFVVWFQSPHTAVKDFV
IIPLHTTPETSVKEIDELVEVYTDVKHRWKAENFIFMGDFNAGCSYVPKKAWKNIRLR
TDPRFVWLIGDQEDTTVKKSTNCAYDRIVLRGQEIVSSVVPKSNSVFDFQKAYKLTEE
EALDVSDHFPVEFKLQSSRAFTNSKKSVTLRKKTKSKRS
Albumin-Linker-DNASE1-LIKE 3, Isoform 2;
Signal Peptide + Propeptide, Albumin, Flexible Linker, mature
DNASE1-LIKE 3 Isoform 2:
SEQ ID NO: 17
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSMRICSFNVRSFGES
KQEDKNAMDVIVKVIKRCDIILVMEIKDSNNRICPILMEKLNREKLVSVKRSYHYHDY
QDGDADVFSREPFVVWFQSPHTAVKDFVIIPLHTTPETSVKEIDELVEVYTDVKHRWK
AENFIFMGDFNAGCSYVPKKAWKNIRLRTDPRFVWLIGDQEDTTVKKSTNCAYDRIVL
RGQEIVSSVVPKSNSVFDFQKAYKLTEEEALDVSDHFPVEFKLQSSRAFTNSKKSVTL
RKKTKSKRS
Albumin-Linker-DNASE2A;
Signal Peptide + Propeptide, Albumin, Flexible Linker, mature
DNASE2A:
SEQ ID NO: 18
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSCYGDSGQPVDWFVV
YKLPALRGSGEAAQRGLQYKYLDESSGGWRDGRALINSPEGAVGRSLQPLYRSNTSQL
AFLLYNDQPPQPSKAQDSSMRGHTKGVLLLDHDGGFWLVHSVPNFPPPASSAAYSWPH
SACTYGQTLLCVSFPFAQFSKMGKQLTYTYPWVYNYQLEGIFAQEFPDLENVVKGHHV
SQEPWNSSITLTSQAGAVFQSFAKFSKFGDDLYSGWLAAALGTNLQVQFWHKTVGILP
SNCSDIWQVLNVNQTAFPGPAGPSFNSTEDHSKWCVSPKGPWTCVGDMNRNQGEEQRG
GGTLCAQLPALWKAFQPLVKNYQPCNGMARKPSRAYKI
Albumin-Linker-DNASE2B;
Signal Peptide + Propeptide, Albumin, Flexible Linker, mature
DNASE2B:
SEQ ID NO: 19
MKWVTFISLLFLFSSAYSRGVFRRDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKD
VFLGMFLYEYARRHPDYSVVLLLRLAKTYETTLEKCCAAADPHECYAKVFDEFKPLVE
EPQNLIKQNCELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFSALEVDETYV
PKEFNAETFTFHADICTLSEKERQIKKQTALVELVKHKPKATKEQLKAVMDDFAAFVE
KCCKADDKETCFAEEGKKLVAASQAALGLGGGGSGGGGSGGGGSATISCRNEEGKAVD
WFTFYKLPKRQNKESGETGLEYLYLDSTTRSWRKSEQLMNDTKSVLGRTLQQLYEAYA
SKSNNTAYLIYNDGVPKPVNYSRKYGHTKGLLLWNRVQGFWLIHSIPQFPPIPEEGYD
YPPTGRRNGQSGICITFKYNQYEAIDSQLLVCNPNVYSCSIPATFHQELIHMPQLCTR
ASSSEIPGRLLTTLQSAQGQKFLHFAKSDSFLDDIFAAWMAQRLKTHLLTETWQRKRQ
ELPSNCSLPYHVYNIKAIKLSRHSYFSSYQDHAKWCISQKGTKNRWTCIGDLNRSPHQ
AFRSGGFICTQNWQIYQAFQGLVLYYESCK
SEQ ID NO: 20
(Intentionally left blank)
SEQ ID NO: 21
(Intentionally left blank)
SEQ ID NO: 23
(Intentionally left blank)
SEQ ID NO: 24
(Intentionally left blank)
SEQ ID NO: 25
(Intentionally left blank)
SEQ ID NO: 26
(Intentionally left blank)
SEQ ID NO: 27
(Intentionally left blank)
SEQ ID NO: 28
(Intentionally left blank)

SEQ ID NO: 29
(Intentionally left blank)
DNASE1L3, Q282_S305delinksK (Signal Peptide; Mature Protein):
SEQ ID NO: 30
MSRELAPLLLLLLSIHSALAMRICSFNVRSFGESKQEDKNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNRLRTDPRFVWLIGDQEDTTVKKSINCAY
DRIVLRGQEIVSSVVPKSNSVFDFQKAYKLTEEEALDVSDHFPVEFKLK
Murine DNase1L3 (O55070): Amino acid sequence (Signal
Peptide; Mature Protein):
SEQ ID NO: 31
MSLHPASPRLASLLLFILALHDTLALRLCSFNVRSFGASKKENHEAMDIIVKIIKRCD
LILLMEIKDSSNNICPMLMEKLNGNSRRSTTYNYVISSRLGRNTYKEQYAFVYKEKLV
SVKTKYHYHDYQDGDTDVFSREPFVVWFHSPFTAVKDFVIVPLHTTPETSVKEIDELV
DVYTDVRSQWKTENFIFMGDFNAGCSYVPKKAWQNIRLRTDPKFVWLIGDQEDTTVKK
STSCAYDRIVLCGQEIVNSVVPRSSGVFDFQKAYDLSEEEALDVSDHFPVEFKLQSSR
AFTNNRKSVSLKKRKKGNRS
Rat DNase1L3 (O89107): Amino acid sequence (Signal Peptide;
Mature Protein):
SEQ ID NO: 32
MSLYPASPYLASLLLFILALHGALSLRLCSFNVRSFGESKKENHNAMDIIVKIIKRCD
LILLMEIKDSNNNICPMLMEKLNGNSRRSTTYNYVISSRLGRNTYKEQYAFLYKEKLV
SVKAKYLYHDYQDGDTDVFSREPFVVWFQAPFTAAKDFVIVPLHTTPETSVKEIDELA
DVYTDVRRRWKAENFIFMGDFNAGCSYVPKKAWKNIRLRTDPNFVWLIGDQEDTTVKK
STSCAYDRIVLRGQEIVNSVVPRSSGVFDFQKAYELSEEEALDVSDHFPVEFKLQSSR
AFTNSRKSVSLKKKKKGSRS
Chimpanzee DNase1L3 (H2QMU7): Amino acid sequence (Signal
Peptide; Mature Protein):
SEQ ID NO: 33
MSRELTPLLLLLLSIHSTLALRICSFNVRSFGESKQEDQNAMDVIVKVIKRCDIILVM
EIKDSNNRICPILMEKLNRNSRRGITYNYVISSRLGRNTYKEQYAFLYKEKLVSVKRS
YHYHDYQDGDADVFSREPFVVWFQSPHTAVKDFVIIPLHTTPETSVKEIDELVEVYTD
VKHRWKAENFIFMGDFNAGCSYVPKKAWKNIRLRTDPRFVWLIGDQEDTTVKKSINCA
YDRIVLRGQEIVSSVVPKSNSVFDFQKAYKLTEEEALDVSDHFPVEFKLQSSRAFTNS
KKSVTLRKKTKSKRS
Olive baboon DNase1L3 (A0A2I3NFJ3): Amino acid sequence
(Signal Peptide; Mature Protein):
SEQ ID NO: 34
MSQELAPLLLLLLSIHSALALRICSFNVRSFGESKQEDQNAMDVIVKVIKRCDIMLLM
EIKDSNNRICPVLMEKLNGNSRRGIMYNYVISSRLGRNTYKEQYAFLYKEKLVSVKRS
YHYHDYQDGDVDVFSREPFVVWFQSPHTAVKDFVIIPLHTTPETSVKEIDELVDVYMD
MKHRWKAENFIFMGDFNAGCSYVPKKAWKNIRLRTDPRFVWLIGDQEDTTVKRSTKCA
YDRIVLRGQEIVSSVVPKSNSVFDFQKAYKLTEEEALDVSDHFPVEFKLQSSRAFTNS
KKSVTVRKKTKSKRS
Rabbit DNase1L3 (A0A2I3NFJ3): Amino acid sequence (Signal
Peptide; Mature Protein):
SEQ ID NO: 35
MSLGMSPASLLLLLLCLHGALALKLCSFNVRSFGYSKRENRQAMDVIVKIIKRCDIIL
LMEIKDSNNMICPTLMEKLNGNSRRGITYNYVISSRLGRNVYKEQYAFLYKEKLVTVK
KNYLYHDYEAGDADAFSREPYVVWFQSPFTAVKDFVIVPLHTSPEASVKEIDELVDVY
MDVKRRWNAENFIFMGDFNAGCSYVPKKAWKNIRLRTDPRFVWLIGDEEDTTVKKSTS
CAYDRIVLRGQDIIRSVVPDSNGVFDFRKAYKLTEEEALDVSDHFPVEFKLQSSTAFT
NSKKSVQPRKKAKAKRS
Dog DNase1L3 (F1P9C1): Amino acid sequence (Signal Peptide;
Mature Protein):
SEQ ID NO: 36
MPRLPAFLLFLLLSISSALALRLCSFNVRSFGGAKRENKNAMDVIVKVIKRCDIILLM
EVKDSNNMICPTLLEKLNGNSRRGIKYNYVISSRLGRNTYKEQYAFLYKEKLVSVKKY
YLYHDYQAGDADVFSREPFVVWFQSPFTAVKDFVIVPLHTTPEASVKEIDELVDVYLD
VKRRWKAENFIFMGDFNAGCSYVPKKAWKIIRLRTDPGFVWLIGDQEDTTVKSSTHCA
YDRIVLRGPEIIRSVVPRSNSTFDFQKAFLLTEEEALNVSDHFPVEFKLQSSRAFTNS
KKSISPKKKKVRHP
Pig DNase1L3 (A0A287B132): Amino acid sequence (predicted
Signal Peptide; Mature Protein):
SEQ ID NO: 37
MSQLLVSLMLLLLSTHSSLALRICSFNVRSFGESKKANCNAMDVIVKVIKRCDIILLM
EIKDSNNMICPTLMEKLNGNSRRSVTYNYVISSRLGRNTYKEQYAFLYKEKLVSVKKS
YLYHDYQSGDADVFSREPFVVWFQSPYTAVKDFVIIPLHTTPETSVKEIDELVDVYLD
VKRRWEAENFIFMGDFNAGCSYVPKKAWKNIRLRTDPMFIWLIKDQEDTTVKKSINCA
YDRIVLRGQEIVSSVVPGSNSIFDFQKAYRLTEEKVRLSFCLSVSPSGEDGVVSPRGI
QATTGDTLGHLTLSFKANDSLT
Guinea pig DNase1L3 (A0A286XK50): Amino acid sequence
(Signal Peptide; Mature Protein):
SEQ ID NO: 38
MSQTRPSLLLLLLAIHGALALKLCSFNVRSFGESKKQNQNAMDVIVKIIKRCDLMLLM
EIKDSHNRICPMLMEKLNGNSRRGTTYNYVISSRLGRNTYKEQYAFLYKEKLVTVKDN
YLFHDEDADVFSREPYVVWFQSPHTAVKDFVIVPLHTTPETSVKEIDELADVYTDVQR
QWKVANFIFMGDFNAGCSYVPKKAWKNIRLRTDPKFVWLIADDEDTTVKKSTSCAYDR
IVLRGQEIVNSVVPNSNGVFDFQKAYQLSEEQALEVSDHFPVEFKLQSERAFTNNKKS
VSLKKKKKANRS
Cow DNase1L3 (F1MGQ1): Amino acid sequence (Signal Peptide;
Mature Protein):
SEQ ID NO: 39
MPLPLACLLLLLLSTHSALALKICSFNVRSFGESKKANCNAMDVIVKVIKRCDIILLM
EIKDSSNRICPTLMEKLNGNSRKGITYNYVISSRLGRNTYKEQYAFLYKEKLVSVKQS
YLYHDYQAGDADVFSREPFVVWFQSPYTAVKDFVIVPLHTTPETSVREIDELADVYTD
VKRRWNAENFIFMGDFNAGCSYVPKKAWKDIRLRTDPKFVWLIGDQEDTTVKKSINCA
YDRIVLRGQNIVNSVVPQSNLVFDFQKAYRLSESKALDVSDHFPVEFKLQSSRAFTNS
KKSVSSKKKKKTSHA
Elephant DNase1L3 (G3SXX1) : Amino acid sequence (Signal
Peptide; Mature Protein):
SEQ ID NO: 40
RSARMSQSLPALLLLLLLSVHGTLALRVCSFNVRSFGETKRENQKVMDIIVKIIKRCD
IMLLMEIKDSNNRICPMLLKRLNGNSRRGIKYNYVISPRLGRNAYKEQYAFLYMEKLL
SVKKSYVYGDNQNGDADVFSREPFVTWFQSPHTAVKDFVIVPLHTTPETSIKEIDELV
DVYMDVKKRWNAQNFIFMGDFNAGCSYVPKKSWRNIRLRTDPGFVWLIGDQEDTTVKE
STNCAYDRIVLRGQIISSVVPNSNSIFNFQKAYELSEEEALNISDHFPVEFKLQSSRA
IINSKKSVSPKKKKKAKSS

LINKER SEQUENCES
SEQ ID NO: 41
GGGGS
SEQ ID NO: 42
GGGGSGGGGSGGGGS
SEQ ID NO: 43
APAPAPAPAPAPAP
SEQ ID NO: 44
AEAAAKEAAAKA
SEQ ID NO: 45
SGGSGSS
SEQ ID NO: 46
SGGSGGSGGSGGSGSS
SEQ ID NO: 47
SGGSGGSGGSGGSGGSGGSGGSGGSGGSGS
SEQ ID NO: 48
GGSGGSGGSGGSGGSGGSGGSGGSGGSGS

ACTIVATABLE LINKER SEQUENCES
FXIIa-susceptible linker (Factor XI peptide):
SEQ ID NO: 49
CTTKIKPRIVGGTASVRGEWPWQVT
FXIIa-susceptible linker (Prekallikrein peptide):
SEQ ID NO: 50
STRIVGG
FXIIa-susceptible linker (Prekallikrein peptide):
SEQ ID NO: 51
VCTTKTSTRIVGGTNSSWGEWPWQVS

1. A method for making a DNASE therapeutic composition for treating a disorder associated with pathological levels of extracellular chromatin or NETs, the method comprising:
evaluating a plurality of extracellular DNASE variants having building block substitutions and/or half-life extension moiety for one or more characteristics selected enzymatic activity, nucleic acid substrate preference, potential for recombinant expression in prokaryotic or eukaryotic host cells, immunogenic potential in humans and animals, and pharmacodynamics in animal models;
selecting a DNASE variant having a desired enzymatic, physical, immunological and/or pharmacodynamics profile, and
formulating the selected DNASE variant for administration to a patient.

2. The method of claim 1, wherein at least 5, or at least 10, or at least 20, or at least 50 extracellular DNASE variants are evaluated.

3. The method of claim 2, wherein the variants are selected from one or more of D1 variants, D1L1 variants, D1L2 variants, D1L3 isoform 1 variant, D1L3 isoform 2 variants, D2A variants, and D2B variants.

4. The method of claim 3, wherein the method evaluates one or more D1L1, D1L2, D1L3, D1L3-2, D2A, D2B, or D1 variants having a building block substitution, fusion or conjugation to a half-life extension moiety, and/or substitution from a non-human D1L1, D1L2, D1L3, D1L3-2, D2A, D2B, or D1, respectively.

5.-12. (canceled)

13. The method of claim 3, wherein at least one D1 variant has a building block substitution selected from human D1L1, D1L2, D1L3, or D1L3-2.

14.-18. (canceled)

19. The method of claim 4, wherein at least one D1L1 variant has a building block substitution selected from human D1, D1L2, D1L3, or D1L3-2.

20.-25. (canceled)

26. The method of claim 4, wherein at least one D1L2 variant has a building block substitution selected from human D1, D1L1, D1L3, or D1L3-2.

27.-32. (canceled)

33. The method of claim 4, wherein at least one D1L3 variant has a building block substitution selected from human D1, D1L1, or D1L2.

34.-39. (canceled)

40. The method of claim 4, wherein at least one D1L3-2 variant has a building block substitution selected from human D1, D1L1, or D1L2.

41.-45. (canceled)

46. The method of claim 4, wherein at least one D2A variant has a building block substitution selected from human D2B.

47.-48. (canceled)

49. The method of claim 4, wherein at least one D2B variant has a building block substitution selected from human D2A.

50. The method of claim 1, wherein the variants comprise an N-terminal or C-terminal fusion to a half-life extending moiety.

51.-52. (canceled)

53. The method of claim 1, wherein the variants comprise variants with one or more polyethylene glycol (PEG) moieties.

54. The method of claim 1, wherein the DNASE variants are evaluated in assays for: altered properties, including altered pH and temperature optimum, requirement for divalent cations for enzymatic activity, mechanisms of enzymatic inhibition, substrate affinity and specificity; localization upon secretion; localization signals; glycosylation sites; disulfide-bonds and unpaired cysteines; compatibility with in vitro expression systems; compatibility with fusion carriers; compatibility with purification methods; toxicological profile; tissue penetration; pharmacokinetics; and pharmacodynamics.

55.-62. (canceled)

63. The method of claim 1, wherein the selected DNASE variant is formulated for topical, parenteral, or pulmonary administration.

64. (canceled)

65. A method for treating a subject in need of extracellular DNA degradation, extracellular chromatin degradation, extracellular trap (ET) degradation and/or neutrophil extracellular trap (NET) degradation, the method comprises administering a therapeutically effective amount of the DNASE variant formulated in accordance with claim 1.

66. A DNASE variant or pharmaceutical composition thereof, the DNASE variant comprising one or more building block substitutions and/or an N-terminal fusion of human albumin and a linker amino acid sequence.

67. The DNASE variant of claim 66, wherein the variant is a D1 variant comprising an amino acid sequence that is at least 80% identical to the enzyme defined by SEQ ID NO: 1.

68.-110. (canceled)

111. A method for treating a subject in need of extracellular DNA degradation, extracellular chromatin degradation, extracellular trap (ET) degradation and/or neutrophil extracellular trap (NET) degradation, the method comprises administering a therapeutically effective amount of the DNASE variant or pharmaceutical composition thereof of claim 66.


SEQUENCE LISTING (51 sequences)

SEQ ID NO: 1 - protein, 282 aa, Homo sapiens [sequence omitted]

SEQ ID NO: 2 - protein, 302 aa, Homo sapiens
MHYPTALLFLILANGAQAFRICAFNAQRLTLAKVAREQVMDTLVRILARCDIMVLQEVVDSSGSAIPLLLRELNRFDGSGPYSTLSSPQLGRSTYMETYVYFYRSHKTQVLSSYVYNDEDDVFAREPFVAQFSLPSNVLPSLVLVPLHTTPKAVEKELNALYDVFLEVSQHWQSKDVILLGDFNADCASLTKKRLDKLELRTEPGFHWVIADGEDTTVRASTHCTYDRVVLHGERCRSLLHTAAAFDFPTSFQLTEEEALNISDHYPVEVELKLSQAHSVQPLSLTVLLLLSLLSPQLCPAA

SEQ ID NO: 3 - protein, 299 aa, Homo sapiens
MGGPRALLAALWALEAAGTAALRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 4 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 5 - protein, 275 aa, Homo sapiens
MSRELAPLLLLLLSIHSALAMRICSFNVRSFGESKQEDKNAMDVIVKVIKRCDIILVMEIKDSNNRICPILMEKLNREKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 6 - protein, 360 aa, Homo sapiens
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 7 - protein, 361 aa, Homo sapiens
MKQKMMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 8 - protein, 30 aa, Homo sapiens
KLSQAHSVQPLSLTVLLLLSLLSPQLCPAA

SEQ ID NO: 9 - protein, 35 aa, Homo sapiens
SAPGTGERAPPLPSRRALTPPPLPAAAQNLVLIPL

SEQ ID NO: 10 - protein, 23 aa, Homo sapiens
SSRAFTNSKKSVTLRKKTKSKRS

SEQ ID NO: 11 - protein, 31 aa, Homo sapiens
RNSRRGITYNYVISSRLGRNTYKEQYAFLYK

SEQ ID NO: 12 - protein, 609 aa, Homo sapiens [sequence omitted]

SEQ ID NO: 13 - protein, 884 aa, synthetic construct
  REGION 1..884: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 14 - protein, 908 aa, synthetic construct
  REGION 1..908: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSFRICAFNAQRLTLAKVAREQVMDTLVRILARCDIMVLQEVVDSSGSAIPLLLRELNRFDGSGPYSTLSSPQLGRSTYMETYVYFYRSHKTQVLSSYVYNDEDDVFAREPFVAQFSLPSNVLPSLVLVPLHTTPKAVEKELNALYDVFLEVSQHWQSKDVILLGDFNADCASLTKKRLDKLELRTEPGFHWVIADGEDTTVRASTHCTYDRVVLHGERCRSLLHTAAAFDFPTSFQLTEEEALNISDHYPVEVELKLSQAHSVQPLSLTVLLLLSLLSPQLCPAA

SEQ ID NO: 15 - protein, 902 aa, synthetic construct
  REGION 1..902: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSLRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 16 - protein, 909 aa, synthetic construct
  REGION 1..909: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 17 - protein, 879 aa, synthetic construct
  REGION 1..879: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSMRICSFNVRSFGESKQEDKNAMDVIVKVIKRCDIILVMEIKDSNNRICPILMEKLNREKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 18 - protein, 966 aa, synthetic construct
  REGION 1..966: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 19 - protein, 958 aa, synthetic construct
  REGION 1..958: note = Synethetic Sequence
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 20-29 - skipped (10 entries)

SEQ ID NO: 30 - protein, 282 aa, synthetic construct
  REGION 1..282: note = Synthetic Sequence
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLK

SEQ ID NO: 31 - protein, 310 aa, Mus musculus
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRS

SEQ ID NO: 32 - protein, 310 aa, Rattus norvegicus
MSLYPASPYLASLLLFILALHGALSLRLCSFNVRSFGESKKENHNAMDIIVKIIKRCDLILLMEIKDSNNNICPMLMEKLNGNSRRSTTYNYVISSRLGRNTYKEQYAFLYKEKLVSVKAKYLYHDYQDGDTDVFSREPFVVWFQAPFTAAKDFVIVPLHTTPETSVKEIDELADVYTDVRRRWKAENFIFMGDFNAGCSYVPKKAWKNIRLRTDPNFVWLIGDQEDTTVKKSTSCAYDRIVLRGQEIVNSVVPRSSGVFDFQKAYELSEEEALDVSDHFPVEFKLQSSRAFTNSRKSVSLKKKKKGSRS

SEQ ID NO: 33 - protein, 305 aa, Pan troglodytes
MSRELTPLLLLLLSIHSTLALRICSFNVRSFGESKQEDQNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 34 - protein, 305 aa, Papio anubis
MSQELAPLLLLLLSIHSALALRICSFNVRSFGESKQEDQNAMDVIVKVIKRCDIMLLMEIKDSNNRICPVLMEKLNGNSRRGIMYNYVISSRLGRNTYKEQYAFLYKEKLVSVKRSYHYHDYQDGDVDVFSREPFVVWFQSPHTAVKDFVIIPLHTTPETSVKEIDELVDVYMDMKHRWKAENFIFMGDFNAGCSYVPKKAWKNIRLRTDPRFVWLIGDQEDTTVKRSTKCAYDRIVLRGQEIVSSVVPKSNSVFDFQKAYKLTEEEALDVSDHFPVEFKLQSSRAFTNSKKSVTVRKKTKSKRS

SEQ ID NO: 35 - protein, 307 aa, Oryctolagus sp.
MSLGMSPASLLLLLLCLHGALALKLCSFNVRSFGYSKRENRQAMDVIVKIIKRCDIILLMEIKDSNNMICPTLMEKLNGNSRRGITYNYVISSRLGRNVYKEQYAFLYKEKLVTVKKNYLYHDYEAGDADAFSREPYVVWFQSPFTAVKDFVIVPLHTSPEASVKEIDELVDVYMDVKRRWNAENFIFMGDFNAGCSYVPKKAWKNIRLRTDPRFVWLIGDEEDTTVKKSTSCAYDRIVLRGQDIIRSVVPDSNGVFDFRKAYKLTEEEALDVSDHFPVEFKLQSSTAFTNSKKSVQPRKKAKAKRS

SEQ ID NO: 36 - protein, 304 aa, Canis sp.
MPRLPAFLLFLLLSISSALALRLCSFNVRSFGGAKRENKNAMDVIVKVIKRCDIILLMEVKDSNNMICPTLLEKLNGNSRRGIKYNYVISSRLGRNTYKEQYAFLYKEKLVSVKKYYLYHDYQAGDADVFSREPFVVWFQSPFTAVKDFVIVPLHTTPEASVKEIDELVDVYLDVKRRWKAENFIFMGDFNAGCSYVPKKAWKIIRLRTDPGFVWLIGDQEDTTVKSSTHCAYDRIVLRGPEIIRSVVPRSNSTFDFQKAFLLTEEEALNVSDHFPVEFKLQSSRAFTNSKKSISPKKKKVRHP

SEQ ID NO: 37 - protein, 312 aa, Sus sp.
MSQLLVSLMLLLLSTHSSLALRICSFNVRSFGESKKANCNAMDVIVKVIKRCDIILLMEIKDSNNMICPTLMEKLNGNSRRSVTYNYVISSRLGRNTYKEQYAFLYKEKLVSVKKSYLYHDYQSGDADVFSREPFVVWFQSPYTAVKDFVIIPLHTTPETSVKEIDELVDVYLDVKRRWEAENFIFMGDFNAGCSYVPKKAWKNIRLRTDPMFIWLIKDQEDTTVKKSTNCAYDRIVLRGQEIVSSVVPGSNSIFDFQKAYRLTEEKVRLSFCLSVSPSGEDGVVSPRGIQATTGDTLGHLTLSFKANDSLT

SEQ ID NO: 38 - protein, 302 aa, Cavia sp.
MSQTRPSLLLLLLAIHGALALKLCSFNVRSFGESKKQNQNAMDVIVKIIKRCDLMLLMEIKDSHNRICPMLMEKLNGNSRRGTTYNYVISSRLGRNTYKEQYAFLYKEKLVTVKDNYLFHDEDADVFSREPYVVWFQSPHTAVKDFVIVPLHTTPETSVKEIDELADVYTDVQRQWKVANFIFMGDFNAGCSYVPKKAWKNIRLRTDPKFVWLIADDEDTTVKKSTSCAYDRIVLRGQEIVNSVVPNSNGVFDFQKAYQLSEEQALEVSDHFPVEFKLQSERAFTNNKKSVSLKKKKKANRS

SEQ ID NO: 39 - protein, 305 aa, Bos sp.
MPLPLACLLLLLLSTHSALALKICSFNVRSFGESKKANCNAMDVIVKVIKRCDIILLMEIKDSSNRICPTLMEKLNGNSRKGITYNYVISSRLGRNTYKEQYAFLYKEKLVSVKQSYLYHDYQAGDADVFSREPFVVWFQSPYTAVKDFVIVPLHTTPETSVREIDELADVYTDVKRRWNAENFIFMGDFNAGCSYVPKKAWKDIRLRTDPKFVWLIGDQEDTTVKKSTNCAYDRIVLRGQNIVNSVVPQSNLVFDFQKAYRLSESKALDVSDHFPVEFKLQSSRAFTNSKKSVSSKKKKKTSHA

SEQ ID NO: 40 - protein, 309 aa, Elephantis sp.
RSARMSQSLPALLLLLLLSVHGTLALRVCSFNVRSFGETKRENQKVMDIIVKIIKRCDIMLLMEIKDSNNRICPMLLKRLNGNSRRGIKYNYVISPRLGRNAYKEQYAFLYMEKLLSVKKSYVYGDNQNGDADVFSREPFVTWFQSPHTAVKDFVIVPLHTTPETSIKEIDELVDVYMDVKKRWNAQNFIFMGDFNAGCSYVPKKSWRNIRLRTDPGFVWLIGDQEDTTVKESTNCAYDRIVLRGQIISSVVPNSNSIFNFQKAYELSEEEALNISDHFPVEFKLQSSRAITNSKKSVSPKKKKKAKSS

SEQ ID NO: 41 - protein, 5 aa, synthetic construct
  REGION 1..5: note = Synthetic Sequence
GGGGS

SEQ ID NO: 42 - protein, 15 aa, synthetic construct
  REGION 1..15: note = Synthetic Sequence
GGGGSGGGGSGGGGS

SEQ ID NO: 43 - protein, 14 aa, synthetic construct
  REGION 1..14: note = Synthetic Sequence
APAPAPAPAPAPAP

SEQ ID NO: 44 - protein, 12 aa, synthetic construct
  REGION 1..12: note = Synthetic Sequence
AEAAAKEAAAKA

SEQ ID NO: 45 - protein, 7 aa, synthetic construct
  REGION 1..7: note = Synthetic Sequence
SGGSGSS

SEQ ID NO: 46 - protein, 16 aa, synthetic construct
  REGION 1..16: note = Synthetic Sequence
SGGSGGSGGSGGSGSS

SEQ ID NO: 47 - protein, 30 aa, synthetic construct
  REGION 1..30: note = Synthetic Sequence
SGGSGGSGGSGGSGGSGGSGGSGGSGGSGS

SEQ ID NO: 48 - protein, 29 aa, synthetic construct
  REGION 1..29: note = Synthetic Sequence
GGSGGSGGSGGSGGSGGSGGSGGSGGSGS

SEQ ID NO: 49 - protein, 25 aa, Homo sapiens
CTTKIKPRIVGGTASVRGEWPWQVT

SEQ ID NO: 50 - protein, 7 aa, Homo sapiens
STRIVGG

SEQ ID NO: 51 - protein, 26 aa, Homo sapiens
VCTTKTSTRIVGGTNSSWGEWPWQVS